(19) United States
(12) Patent Application Publication
(10) Pub. No.: US 2022/0166621 A1
(43) Pub. Date: May 26, 2022

(54) ARRANGEMENT FOR ENCRYPTED EXCHANGE OF PERSONAL MEDICAL AND FINANCIAL DATA

(71) Applicant: GeneJunction Pte. Ltd., Singapore (SG)

(72) Inventors: Rachna Kairon, Kuala Lumpur (MY); Rakesh Kumar Aggarwal, Singapore (SG)

(21) Appl. No.: 17/442,014

(22) PCT Filed: Mar. 25, 2020
(86) PCT No.: PCT/SG2020/050165

§ 371 Date: Sep. 22, 2021

(30) Foreign Application Priority Data
Mar. 25, 2019 (SG) 10201902656U

(51) Int. Cl. H04L 9/32; G16H 10/60

(52) U.S. Cl. CPC H04L 9/32; G16H 10/60

(57) ABSTRACT

An arrangement for encrypted exchange of personal medical and financial data comprises a mass enrollment center computing device, a subscriber computing device, a web platform server device, and a payment provider server device. The web platform server device creates a temporary link site with a temporary link site identifier and forwards the link site identifier to the mass enrollment center computing device and to the subscriber computing device. Pre-determined messages are exchanged between the mass enrollment center computing device and the subscriber computing device, using a computer memory link location for releasing health-related data to the subscriber computing device. The web platform server device, upon reception of a payment signal from the payment provider server device, automatically initiates a transmission of identity information data of at least one subject from the mass enrollment center computing device to the subscriber computing device.

The present specification provides an arrangement for the secret and secure communication by providing a multitude of transmitting and receiving encryption devices that are set up in a particular manner.

Advances in DNA microarray and polymerase chain reaction (PCR) technologies now allow researchers to measure gene expression for thousands of genes at once. However, there is a need for better methods and systems for collecting genomic information, providing access to the information, making use of the information collected, and correlating the information with other participant-related information, such as medical information.

The EP 0167565B1 discloses a cryptographic transmission system with a synchronization feature that detects when a synchronization of an encrypted communication is required and that provides the synchronization in a first transmission direction and in a second transmission direction.

The EP 3155754 B1 discloses a system for providing encryption on a plurality of devices. The system can automatically and securely synchronize a user's file encryption/decryption keys across a plurality of devices, authenticating the user on each device before receiving and processing information from the server necessary to recreate the user's file encryption/decryption keys.

The EP 1583380 B1 discloses a system for a call encryption in a terrestrial trunked radio network with a synchronization feature. A transmit unit (201) transmits a new encryption synchronization message when the elapsed time exceeds the time threshold.

The application provides an arrangement or a system for encrypted exchange of personal medical and financial data. Such data is often personal data and benefits from an enhanced protection by the lawmakers.

No sensitive information can leak out by copying the available information from one single participant, also referred to as communication party or communication partner. The efforts according to the abovementioned publications focus on improving the encryption algorithm, while the system of the present specification seeks to set up the transmitting and receiving encryption devices in such a particular manner that the sensitive personal data is better protected by a distributed and timely staggered way of exchanging communications.

The arrangement according to the present specification comprises at least one mass enrolment center computing device which is operable to store health related data and identity information data of a multitude of subjects. There can be a large number of more than thousand subjects or an even much larger number, such as ten thousand, hundred thousand, or more. While the health-related data comprises health state data according to its type and genetic information data according to its type, both not being critical, the identity information data is highly critical with respect to confidentiality.

In addition to the health state data according to its type and genetic information data according to its type, there is actual health state data and genetic information data of subjects, but the arrangement according to the application does not store this critical actual data of subjects, thereby providing a further security level. The actual health state data and genetic information data of subjects is exchanged only after personal and financial data have been exchanged over the arrangement according to the application.

A subscriber computing device is operable to receive the health-related data and identity information data via a communication interface, which enables an encrypted way of handling the communication over a web platform server device that provides an encrypted communication channel between the subscriber and the mass enrolment center computing device. The communication interface of the subscriber device implements an encrypted transfer protocol. Furthermore, it can implement a graphical user interface.

A payment provider server device receives a payment information from the mass enrolment center computing device and/or from the subscriber computing device and provides a payment signal to the web platform server device that the payment has been received.

A recipient, which receives data from a data base of an MEC or at the web platform pays the MECs through a payment provider in bulk payment. For instance, the bulk payment can by a payment of USD 500 for 100 contributors. It is a two-way transaction which involves (i) a payment to the MEC and (ii) a payment to the health data provider.

As an additional technical feature, upon this transaction an e mail is sent to all linked contributors of the MEC informing them that a transaction has taken place. The MEC can pay individual contributors as per arrangement with respect to the MEC.

The communication channel, which is established by the web platform server device, comprises a temporary link site with a temporary link site identifier in the form of an IP address that can be assigned from a pool of available IP addresses, and a computer memory link location which can comprise a digital memory storage area. The temporary link site can use website technology and messenger functions based on pre-determined messages that can be selected by the subscriber and the subjects.

The temporary link site identifier is forwarded to the mass enrolment center computing device and to the subscriber computing device, and a communication interface is provided which allows the encrypted interchange of pre-determined and not freely generated messages that are related to the health data type and/or to the genetic data type between the mass enrolment center computing device and the subscriber computing device.

The communication interface of the temporary link site uses the computer memory link location for releasing the health-related data to the subscriber computing device, but not yet the identity information. In particular, the communication interface of the temporary link site can provide the abovementioned communication interface of the subscriber computing device.

The web platform server device, only upon reception of a payment signal from the payment provider server device, automatically and without interaction of the subscriber or the subject initiates a transmission of the identity information data of at least one subject from the mass enrollment center computing device to the subscriber computing device, upon which an operator of the subscriber computing device can contact the respective subjects for receiving the detailed real data, in contrast to the type data, wherein type data specifies a type of available data and can refer to exchange categories. The automated procedure to provide the identity information data reduces the risk of fraud and increases the speed of the transactions.

The structure of this arrangement enables a safe, secret and encrypted exchange of medical related data, and it is set up in a particular and secure manner.

The communication interface can provide an automatic verification of a matching of health-related data with a predetermined message of the subscriber computing device, upon which the subscriber computing device is triggered to send a payment information to the payment provider server device. This enhances the speed of a handling of a large multitude of subject data before the data expires, for example by a progress of the specific diseases or a passing of the subjects.

The web platform server device stores a first identifier, for example in the form of an IP address of the mass enrolment center computing device, and a second identifier of the subscriber computing device, wherein the communication interface is maintained between the first identifier or IP address and the second identifier first identifier or IP address. This increases the security of the arrangement.

In a further variant, the web platform server device deletes the temporary link site and the computer memory link location after transmission of the identity information data of the least one subject from the mass enrollment center computing device to the subscriber computing device, thereby increasing the security of the arrangement. In particular, deleting the computer memory link location can refer to deleting or overwriting the computer memory which contains the link location.

According to a further embodiment of the arrangement, the web platform server device is furthermore operative to receive identification data of a referred person from a referring subject which is registered at the mass enrolment center computing device.

Upon registration of the referred person, gene credits which amount to values or credit points stored in a computer memory and which are equivalent to monetary value, are credited to an account of the referring subject.

According to a further embodiment of the arrangement, the web platform server is furthermore operative to attribute a third-party verification service to one or more registered subjects which are registered by the web platform server. The attribution of the third-party verification service is randomized, for example by randomly assigning an id of the verification data to the one or more registered subjects.

Thereby, a third-party evaluation can be more accurate and objective as compared a situation in which the third party verification service or another participant chooses the data to be evaluated.

Furthermore, the third-party verification service is caused to automatically verify health related data of the one or more registered subjects, for example by sending a notification message to the third-party verification service, wherein the notification message contains a link to the health related data to be evaluated or by sending anonymized data to be evaluated to the third-party verification service.

The health-related data is filtered according to exchange categories or digital gene assets that are individually specified by the one or more subjects, thereby allowing the subjects a control over the data that they are willing to provide.

According to a further embodiment, the web server computing device is furthermore operative to receive a health exchange category of health-related data from one or more subject and to store the health exchange categories of the one or more subjects individually for each subject.

Furthermore, the web server computing device is operative to provide access to health-related data according to the stored health exchange categories.

The health exchange categories of the health-related data are selected from genetic data relating to biochemical genetics, molecular genetics, clinical cytogenetics, preimplantation genetic screening or non-invasive prenatal testing, molecular diagnostics of infectious diseases, clinical trials, sharing of an existing specimen, providing a new specimen, radiology procedures, laboratory reports, electronic medical records and health related data retrieved from wearable devices.

Thereby a means is provided to allow the subjects to control the access to their data according to their selected exchange category. Furthermore, a reward for the use of subjects' health data in terms of gene credits can be provided according to the selected exchange categories.

According to a further embodiment of the arrangement, the web server computing device is furthermore operative to store health related data of one or more subject, to receive a first evaluation from experts, and advisory committee or other third party verification services of the health-related data 19/1 based on the health-related data. Based on exchange categories chosen by the individual subjects and after receiving the first evaluation, the first evaluation is adjusted based on a demand of the health-related data. The demand can for example refer to a number of queries to the health-related data. Thereby, a value of the health-related data of the subjects can be provided, for example in terms of gene credits or in terms of a monetary value.

In a further aspect, the specification provides a computer implemented method for encrypted exchange of personal medical and financial data. Health related data and identity information data of a multitude of subjects at a mass enrolment center computing device is stored in database.

The health-related data and identity information data is received via a communication interface at a subscriber computing device. An encrypted communication channel is provided between the subscriber computing device and the mass enrolment center computing device by a web platform server device. Thereby, a transfer of sensitive data, such as identity related data can be protected.

A payment information is received at a payment provider server device, and a payment signal is provided to the web platform server device. The provision of the encrypted communication channel by the web platform server device comprises the following steps.

A temporary link site is created with a temporary link site identifier and a computer memory link location. The temporary link site identifier is forwarded to the mass enrolment center computing device and to the subscriber computing device.

The temporary link site provides a communication interface which allows the encrypted interchange of pre-determined messages between the mass enrolment center computing device and the subscriber computing device. The computer memory link location is used for releasing the health-related data to the subscriber computing device. In other words, the health-related data is made accessible by the temporary link site identifier in the computer memory link location.

Upon reception of the payment signal a transmission of the identity information data is initiated of at least one subject from the mass enrolment center computing device to the subscriber computing device.

According to a further embodiment of the method, an automatic verification of a matching of the health-related data with a predetermined message of the subscriber computing device is carried out or provided, and a payment information is sent to the payment provider server device.

According to a further embodiment of the method, a first identifier of the mass enrolment center computing device and a second identifier of the subscriber computing device is stored and the communication interface is maintained between the first identifier and the second identifier.

In a further embodiment of the method the temporary link site and the computer memory link location is deleted after transmission of the identity information data of the least one subject from the mass enrollment center computing device to the subscriber computing device. Thereby a security of the data transfer can be enhanced.

According to a further embodiment, the method comprises receiving identification data of a referred person from a referring subject which is registered at the mass enrolment center computing device. Upon registration of the referred person gene credits such as value or credit points in computer memory which are equivalent to monetary value, are awarded to an account of the referring subject.

According to a further embodiment of the method, which provides a randomized third-party verification, the method further comprises the following steps.

A third-party verification service is attributed to one or more registered subjects which are registered by the web platform server. The attribution of the third-party verification service is randomized, for example by assigning an id of the verification data to the one or more registered subjects.

The third-party verification service is caused to automatically verify health related data of the one or more registered subjects. This can be carried out for example by sending a notification message to the third-party verification service the notification message containing a link to the health related data to be evaluated, sending anonymized data to be evaluated to the third-party verification service, wherein the health related data is filtered according to exchange categories or digital gene assets that are individually specified by the one or more subjects.

According to a further embodiment of the method, a health exchange category of health-related data is received from one or more subject. The health exchange categories of the one or more subjects are stored individually for each subject.

Access to health-related data is provided according to the stored health exchange categories. Those health exchange categories of the health-related data are selected from genetic data relating to biochemical genetics, molecular genetics, clinical cytogenetics, preimplantation genetic Screening or non-invasive prenatal testing, molecular diagnostics of infectious diseases, clinical trials, sharing of an existing specimen, providing a new specimen, radiology procedures, laboratory reports, electronic medical records and health related data retrieved from wearable devices.

Furthermore, the method can provide an evaluation of contributor data. Health related data of one or more subject is stored, a first evaluation is of the health-related data is received over an electronic communication channel from experts, an advisory committee or other third party evaluation services based on the health related data and based on exchange categories chosen by the individual subjects.

After receiving the first evaluation the first evaluation is adjusted based on a demand of the health-related data.

Furthermore, the application discloses a computer-readable digital storage area or computer readable memory which can include multiple physical carriers or a cloud storage are comprising instructions which, when executed by a computer system in an arrangement of computer devices, provides the aforementioned method steps of operating the arrangement of the application.

The following parties to the transactions of the health information exchange system are involved in the collection of personal health data.

“Subjects” may in particular refer to humans, but, in a wider sense, also to other forms of life, such as animals or plants. The subjects can provide information in the form of untested or unconfirmed genetic information, available raw genetic data, previously tested results or reports, available specimens or the potential to provide one, results from participating in basic research or clinical trials and affiliated consented information, among others. In the case of other life forms, the subjects can be represented or their respective information can be entered by a human user, which may then also be referred to as the subject.

Subjects are also referred to as “contributors”. A “contributor” is an individual who registers on the online platform for the fee stated based on different packages. Once registered, they have the choice whether to share or not to share their health information.

“Subscribers” are parties or individuals who wish to link up with a subject. Subscribers often pursue drug discovery and research, development of new treatment modalities, epidemiology, clinical trials, basic research, networking to form a support group, and, in a broader sense, they can build a global genetic bank for mankind and generations to come for the benefit of science.

The subscribers are also referred to as “recipients”. A “recipient” is someone interested in acquiring the health information from the Contributor. A recipient registers for the fee stated based on different packages and is able to filter and search for the health information from the contributors.

There are mainly two groups of recipients: those whose are interested in medical data mining and those who are interested in business data mining. The first group includes pharmaceutical companies, drug makers, medical research centers. They can make use of the data for determining investments, performing targeted clinical trials, medical research, etc. The second group of recipients includes merchants and service providers who aim to discover patterns and relationships in the data in order to make better business decisions, such as developing smarter marketing campaigns, predicting customer loyalty or predicting customer demand and purchasing patterns.

A “mass enrolment center” is defined as an organization, party group or association that has access to multiple subjects who can be enrolled into a personal data collection process. This includes clinics, laboratories, universities, research organization/s etc.

In a further aspect, “mass enrolment centers” (MEC) are organizations that have a pool of people that can be registered as a Contributor such as Clinics and Hospitals who have a patient list. They manage the pool of Contributors and enroll with Recipients for medical research and clinical trials.

“Third-party verifiers” are people who are qualified to verify clinical, genomic or any other personal health data information. These include genetic counsellors, clinicians etc. Thereby, a quality of information can be verified, or a “commodity” being sold on the site. This also provides confirmations to the subscribers regarding the credibility of information. Both “subjects” and “subscribers” may use this verification service.

The subjects may use the verification service to add credibility to their information while posting it. “Subscribers” may trigger a verification after linking to the subjects and on the anonymous information exchange platform prior to the actual transaction and this exchange is available on the anonymous information exchange platform. This can be provided as an optional feature for both parties of the information transaction.

As an added service, “genetic counselling services” can be provided through a link, which is a service for which subjects may pay third-party counsellors through the portal.

A “third-party consultant”, which is also referred to as third-party verifier, is someone who is a qualified clinician, medical counsellor, phlebotomist who provide their professional medical services through counselling and verifying the health and genetic data. Furthermore, the third-party consultant provides medical and digital consults or consultations online.

A “representative” is someone who seeks and approaches the public and register individuals as a Contributor through the go-to-market strategy. The representative is also referred to as “partner” or “allied partner”. The representatives act as a sales force and earn a fee for each contributor they have registered. According to a multi-level marketing model, an agent represents a territory and has a number of allied partners under him. The agents get a fixed percentage of each partners collections or revenue in accordance with a multi-level marketing strategy.

The health information exchange system according to the specification provides a platform for sharing of genetic information or data between “subjects” who “own” this information in the form of raw data, results, reports or specimens or participating in basic research or clinical trials and “subscribers” who need this information. The health information exchange system can meet “link” and “match” requirements of subscribers and subjects to allow this exchange effortlessly, while safeguarding privacy and records of both parties. A payment gate way enables this link.

The health information exchange system according to the specification provides an online portal to link “subjects” who have information with “subscribers” who need this information and, in the process, can build a worldwide network and catalogue of genetic data in conjunction with available and consented clinical information.

The information exchange system provides, among others, a solution for the technical problem of establishing an anonymous information exchange between a subject, which provides medical information about itself, and a subscriber, which is interested to retrieve the medical information.

In further embodiments, the information exchange system also provides a solution for the technical problem of automatically exchanging medical information between the subject and the subscriber and obtaining consent for retrieving the information, for example by using a proxy server, a robot or a program running in the background. Thereby, it is not required that the parties to the communication are permanently online and the information can be exchanged for a great number of subjects and/or subscribers.

Furthermore, the information exchange system provides a technical solution for managing the payment flow in exchange for using the services of the information exchange system and for providing and retrieving the information.

In each of the abovementioned cases, the information exchange system of the present specification provides a solution to the technical problem at hand, as opposed to merely implementing a different solution in which for the technical problem, such as establishing a two-way anonymous connection for the interchange of medical information, does not occur, or in other words, is circumvented.

The information exchange system is adapted to handle a large amount of individual health related information. To this end, the information exchange system provides a mechanism for the automated handling of the information retrieval. The handling of the information retrieval can comprise, among others, the automated obtaining of consent, requesting data that matches to a requirement profile of a client, a filtering and evaluation of obtained data, and determining, whether further information needs to be retrieved.

Purpose/potential applications/value chain: evaluation of a health product/service to be sold with respect to: commercial value, potential improvement. Statistical evaluation with the aim of obtaining knowledge/information relating to treatment efficiency, population statistics, course of disease, epidemiology and so forth.

A mechanism to obtain consent, such as a consent button, provides legal validity of the data, protection against fraud and allows the disclosure of more than the displayed information.

According to one embodiment, the information system provides an online portal to build a worldwide catalogue of previously conducted genetic tests conducted and through this database readily accessible genetic data. Furthermore, the information system provides a payment gateway for procurement of genetic information or data by “subscribers” who need this information from “subjects” who “own” this information in the form of raw data, results, reports or specimens.

The information system can provide a linking and matching between the requirements of subscribers and subjects to allow an efficient health information exchange, while maintaining privacy and records of both parties.

According to one embodiment, the information system does not allow posting or sharing of real time data. The information system provides personal data production, such as defined for example in the “Personal Data Protection Act Guidelines”. According to another embodiment, a third-party system or a data storage system contains reports. For example, a means can be provided to upload these reports to the health data provider website.

Furthermore, the information system provides a mechanism through which a consent to exchange data can be obtained and administrated, such that a posted health information posted is posted through explicit consents of all participants. The information system can facilitate data sharing between two independent parties and provide a listing and collating of available global genomic information.

Moreover, the information system can also enable the subject and the subscriber to mutually and independently assess the value of that information exchange while using this platform only to enable the link and transaction.

Specifically, the health information system can provide a nodal information hub for health information and an adaptable tool for future prognostic diagnostic and preventive medical decisions. In particular, the exchanged health information can comprise genomic data, which is a resource for analysis and data mining, and can lead to development of new technologies and strategies to improve health care. This includes diagnostics, disease specific treatments, drug discovery, and preventive approaches. Data may be shared for individual comparisons, clinical outcomes, clinical trials, research, drug discovery, gene therapy, among others.

Among others, the health information can include results of diagnostic genetic tests, for example tests for single gene disorders, such as sickle cell disease cystic fibrosis, or tests for multifactorial disorders, such as Alzheimer's disease, breast ovarian cancer, colon cancer and hypothyroidism. Furthermore, the health information can also include, among others, the results of carrier tests, genetic Traits, predictive/presymptomatic genetic tests, clinical exome tests, whole exome tests, prenatal genetic tests, non-invasive Prenatal tests, prenatal fetal sexing, a preimplantation Genetic Diagnosis, newborn screening tests, pharmacogenetics, a sampling of blood tissue (biopsy) and other research data.

According to an exemplary embodiment, the information system allows “subjects” who have some form of genetic information regarding themselves to make this information available to “subscribers” who wish to link with them to access the Information for Research or other applications. The information system can provide a link that creates a global platform for Genomic and provide a database of classified genetic information. In particular, the information system can provide a generic classification system to allow easy access of the available information such as “single gene” or “whole genome sequence”.

In a further embodiment, the information system can provide a “genomic stock exchange” or a marketing of other health related information. Thereby, the potential of the web for enabling concepts for amassing buyers and sellers can be exploited. For example, by using a previously created classification code “genetic code” for various genetic disorders that people are buying or selling through our site, the information system can provide a portfolio of genetic stocks. For example, the information system can provide a genomic information exchange by means of which a trading of these “genetic shares” is offered.

The genetic stock refers to digital gene assets provided by a contributor. The digital gene assets, which are also referred to as “exchange categories” or, in other words, categories of data that a contributor is willing to exchange, provide a concept of digital gene rights, whereby a person manages his or her own digital gene assets.

A value of the genetic stock can be determined by experts and an advisory committee of the health data provider. Subsequently, the value may be adjusted based on demand, wherein demand can be defined as a number of subscribers looking for a specific information and value offered. For example, the value can be a benchmark value that is determined by a factor of cost plus a pre-determined percentage.

As mentioned above, a value is assigned to each genetic stock. By way of example, a value of each genetic stock can be based on a volume of links created per month for that stock code and/or on a value of the transactions on that genetic stock for every month. In summary, the value of a genetic stock can be determined by the buyer and the seller based on volume and amount of buy and sell transactions of genetic information through use of the information system.

In a further embodiment, a genomic token is provided, which contains a total number of genomic stock code or digital gene assets plus a value of all genomic stock code. The token can be registered on an information exchange platform to raise funds. The raised fund can be used to provide a free genetic profile to all contributors on the site.

The feature of a genomic stock exchange can make use of the transactions for data which are performed through the web platform of the health data provider. According to one embodiment, each kind of genetic test is provided with a code and the number and values of transactions is codified for each kind of digital gene asset. The exchange category is also referred to as “digital gene asset”. The genomic stock exchange makes use of the allocation of a number and value to each kind of transaction.

According to one business model, every buy or sell transaction of the information system benefits subjects and subscribers by crediting to their accounts a suitable fee from each buy and sell through the information system. Furthermore, the subjects may receive a gene credit amount into their account.

The information system according to the present specification can provide various advantages over existing tools. For example, the information system can provide a sharing of data with researchers and people who need the data to advance science. The information system can provide a more comprehensive database of health information, such as genomic information.

Thereby, the information system can complement existing tools such as whole genome sequencing tools. By providing a financial incentive for the subjects to provide their individual health information, a comprehensive information can be provided worldwide and at a low price.

According to one specific embodiment, the information system provides a free subject enrolment. By enrolling the subject agrees to terms and conditions, which are provided through a graphical user interface of the information system. Similarly, the subscribers which want to obtain individual health information need to agree to terms and conditions, which are displayed via a graphical user interface of the information system. For example, the subscribers may be obliged to refrain from tracing back the individual information, such as by profiling and matching an information flow through internet search engines using the obtained data.

After enrolment, the information system allocates to a subject a unique tag, for example in the form of a bar code, the subject is identified by the tag instead of a user name or any traceable information. Associating the information with a tag instead of aggregating the data can provide several advantages such as being able to trace a change of the data over time and avoiding data duplication and overlap.

According to one embodiment, the medical information exchange system can provide a three pronged “financial model”. Main features of these three aspects of the financial model are summarized below.

According to a first aspect of the financial model, a revenue is obtained through Registration and Transactions. Firstly, subjects, subscribers, mass enrolment centers and third-party verifiers build a network through a web portal or web platform and also add to the base registration fees.

Secondly, subjects and mass enrolment centers are able to build a value for and also monetize their own data in the form of clinical or genetic information through a portal provided by the health information exchange system. This is achieved through the exchange transaction and payment gateway as subscribers pay for this information. The company which operates the web platform, may also impose small fees which shall be declared transparently. Furthermore, the health information exchange system can make use of the concept of so called “gene credits”, according to which credits shall be passed back to each subject based on transactions conducted.

Thirdly, the health information system can furthermore obtain advertising revenues from interested parties which could be pharmaceutical parties, research groups and potentially others when the population of the portal has reached critical mass. Fourthly, services, such counselling and verification can add additional revenue to the subjects with a fee allocated to the health information provider. Furthermore, the health information system can provide a global online health consultation.

According to a second aspect of the financial model, a revenue can be obtained through building value: according to this concept, subjects 12 are made “stakeholders” or “shareholders” in the company. To this effect, the health information system can provide a percentwise owner ship, such as 20%, of the company to be owned by “subjects” once a critical mass has been reached, which may be determined by hits, turnover or other performance indicators, which may be determined automatically and/or with the help of user provided evaluation input.

A progressive recruitment of more and more subjects who believe that “we own our own data and should drive science” is the value proposition in itself whereby the site builds its own value. This can be monetized through “third-party investors”, “listing the company”, “sponsors” and this value is passed back to subjects who have registered on the site.

According to a third aspect of the financial model, a revenue can be obtained through genomic stock exchange. As sufficient volume of information becomes available via the portal and transaction activity commences between the subscribers and subjects, the health information provider can invite interested players to engage in genomic stock trading of various commodities as detailed elsewhere in this summary. By way of example, this invitation may be provided automatically in the form of messages or in the form of user specific information displayed by the health information provider.

In the following a features workflow is outlined that provides features in addition to the above listed features.

The health information exchange system can provide an enrolment of regular and premium subjects. Furthermore, it can provide a global registration. According to this concept of the health information exchange system anybody, whether healthy or diseased can potentially benefit by enrolling on to this site. In order to enable this mass enrolment, the health information exchange system can provide, among others, the two following concepts. Firstly, the health information exchange system can provide a consent proxy for a mass enrolment center. This center could be a clinic, a laboratory, a university, A hospital, an NGO, a third-party service provider, just to name a few examples.

Furthermore, the health information system can target a mass market through the use of social media, by providing apps, for example for android and IPhone, thereby helping to recruit mass numbers though mobile applications. This is especially significant as the health information does not only recruit diseased people but also healthy individuals who become stakeholders or shareholders in the company.

The financial model or payment gateway for transactions of the health information can be characterized, among others by the following features: it allows a one-to-one mapping of communication partners, an anonymous exchange of information, an information exchange on web site using fixed messages, a negotiation on web site, a third-party verification of the information supplied. Furthermore, the health information exchange system can provide a use of e-wallets for direct payments, for example for direct cash payments to contributors.

The web platform 28 can provide a transparency of transaction by declaring service provider fees using VISA Paypal, a Stripe payment or other payment providers as well its own fees thereby ensuring payments to subjects. Herein, a “Stripe” is a payment processor, which supports the electronic transfer of money from a customer's bank or issuing bank into a merchant's bank or acquiring bank as payment for goods or services bought with a credit card. Furthermore, the web platform 28 can provide an automatic pricing while allowing one-time negotiation on site.

Furthermore, the web platform can provide an e-wallet which allows a three-way transaction from recipient to MEC, to the linked contributor and to the health data provider.

In particular, the e-wallet can be a “third-party wallet” or payment channel of a payment provider, for two party transactions. In an extended embodiment, the payments from recipients to the health data provider, the MECs and linked contributors are prorated based on a fixed percentage.

Furthermore, the web platform can provide a global money transfer in exchange for a small fee. This service feature that can be made available to all contributors on this site. To this end, the accounts can be protected, or an existing protection mechanism can be used. For example, providing Gene ID of contributors while transferring monies can provide an added security feature.

The e-wallet allows monies to be exchanged and transferred globally. Since we have a panel of contributors registered on the site, the site has a database that allows recipients to connect with contributors and potentially develop a global money exchange system which is linked to individual gene accounts and thereon their bank accounts which provide standard automated currency exchange rates. Gene Junction can then propose a small fee to allow this function.

Furthermore, the e-wallet can be used to monetize gene credits provided by the health data provider.

According to one embodiment, the financial method features provided by the health information exchange system with respect to a stakeholder/shareholder concept can be characterized as follows. The health information exchange system can provide a “democratization” of data and an ownership of data by the “subjects” themselves, who thereby have the right to enforce their own choice and can benefit not only financially but also in kind. The “subjects” can own the site collectively and can be the engines for its growth.

According to one embodiment, all subjects can be considered as “stakeholders”. To further incentives the growth of the site and to build a financial model around, the users of the site can comprise in particular two categories of subjects Subjects who enroll for free can benefit in two ways i) By earning fees through sale of available information and items and ii) Earning gene credits (for example 1% of each sale) which potentially translate to monies which can allow purchase of a genetic test and/or cash back.

Subjects who enroll for a small proposed fee, such as for example 3 USD can benefit by becoming stakeholders or even premium stakeholders or shareholders in the company.

Premium stakeholders can benefit in three ways. Firstly, they can benefit by earning fees through sale of available information and items. Secondly, they can benefit by earning gene credits, for example 1% of each sale, which potentially translate to monies which can allow a purchase of a genetic test or cash back. Thirdly, premium stakeholders can also benefit by becoming shareholders in the company, as explained below.

Optionally, a predetermined percentage, such as up to 20% of the company equity can be reserved for subjects who subscribe for a fee. This activity shall be activated once the company has reached a pre-determined threshold of premium subjects, such 1 million subjects, enrolled or a pre-determined minimum net revenue target is achieved by the company. Premium subjects are premium stakeholders/shareholders.

This status can be reviewed annually. The predetermined percentage of the company equity value is translated to a monetary value, such as USD or other currencies, and offered as free tests to subjects and mass enrolment centers in equal proportion and in order of registration by timeline. Optionally, the benefit can be monetized, but preferentially, the health information exchange system provides free tests.

According to a further aspect of the financial model, the funds generated through this Portal can provide an engine for its own growth. Among others, a source of funds can be provided by the following means. One source of funds can be obtained through direct transaction between subjects and subscribers, the company earns fees, for example 5% of transaction value. A further source of funds can be obtained by way of registration fees of subscribers, for example USD 5, and registration fees of premium subjects, for example USD 3.

A further source of funds can be provided by levying percentage fees for counselling or third-party verification. For example, the company or health provider can raise a fee of about 3%. A further source of funds can be provided by advertising fees through Market place. The advertising may be provided by the health information exchange system itself or it may also be provided by an advertisement provider, which exchanges information with the health information exchange system.

Moreover, a concept which provides a registration of subjects who are willing to be part of a global “story” and a population of the website with sufficient numbers can in itself be a value proposition. Furthermore, a model which provides, among others, the development of a global platform of genetic data, information exchange, a direct link and of a payment gateway is in its own automated mechanics can create value for itself and attract investors in this concept. A suitable valuation could generate funds which shall be used for the following activities.

As a first activity, the web platform can provide free or highly subsidized genetic tests, for example, whole genome sequencing or whole exome sequencing with basic informatics and interpretation. Thereby, the site can be populated even further with information that “subscribers” need. The larger the valuation, the larger the data base, the larger the valuation of the company gets. This can provide a self-sustaining cycle. The larger the projections, the bigger the chance to double or triple the valuation based on the value created. This effect can lead to exponential growth over a certain time.

As a further activity, the health information exchange system can provide marketing and activities to populate the site globally. Through various social media as well as other direct marketing techniques, which are provided by the health information exchange system, potential subjects and subscribers can be reached, which populate the portal and spread awareness among various interested parties, such as physicians, researchers, pharmaceutical companies, subscribers and Subjects) of the availability and utility of the clinical and genomic information on the portal.

The size of the health information exchange system can be tailored to the needs of the subjects/subscribers. For example, it can be provided for one mass enrolment only or for multiple mass enrolment centers. In this sense the health information exchange system can be decentralized. Furthermore, it can provide an anonymous one-to-one exchange between subjects and subscribers and it can provide a system to distribute financial rewards to the participants.

The health information exchange system can provide a reward mechanism in exchange for the obtained information. Furthermore, it can provide a communication gateway without allowing direct communication between the communication partners.

The health information exchange system can provide a one-to-one information exchange or a reward for the access to the subject's information. Furthermore, it can provide a mechanism by which the person requesting a subject's information is not able to negotiate the reward with the information providing subject. Furthermore, the health information system can provide a rating system which provides individual ratings for the information provided by a subject. The health information exchange system can provide an automated mechanism for remuneration such as by way of direct payment or assigning gene credits.

The health information exchange system of the current specification can provide an automated mechanism to exchange information anonymously between a subject and a subscriber, such as the information exchange system of the present specification. By making the communication anonymous, the subjects and the subscribers are in general not able to communicate with each other directly and are more inclined to pay for the communication facilities provided by the platform.

In contrast to a database with only aggregated or anonymous data, the information exchange system of the present specification allows a one-to-one mapping of a medical information to a subject, which can avoid data duplication and the follow-up of information, as well as an individual payment for the provided information which may also be negotiated individually, without personal data unintentionally being released to one or more of the parties being involved in the transaction process.

The subject matter of the present specification is now explained in further detail with respect to the following Figures in which

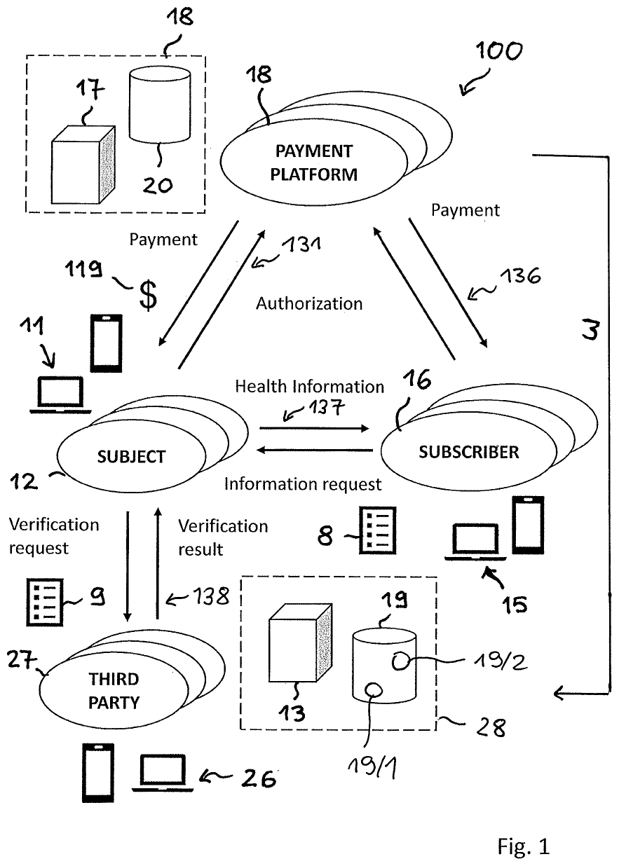

FIG. 1 shows a first embodiment of a medical information exchange system,

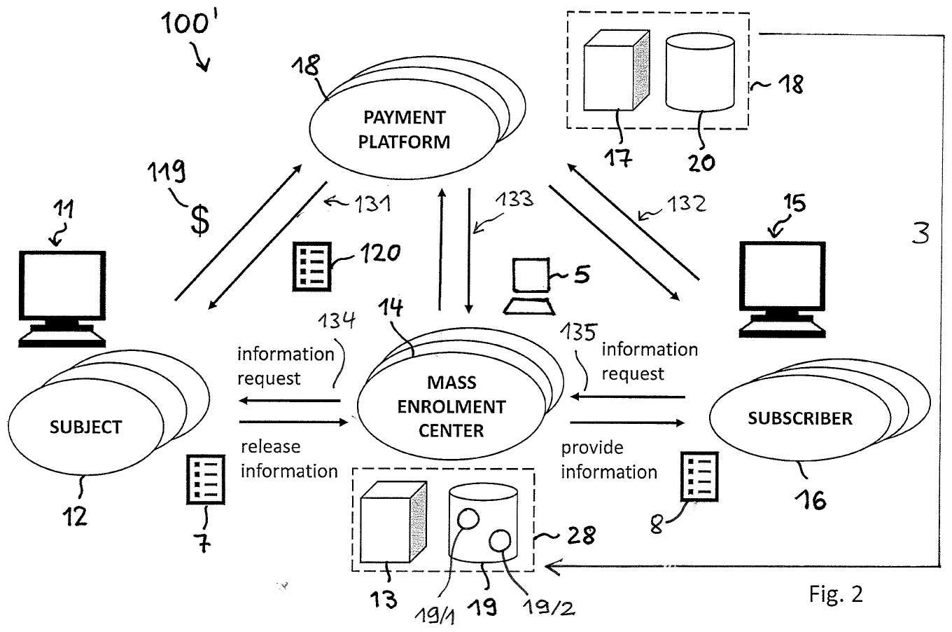

FIG. 2 shows a second embodiment of a medical information exchange system,

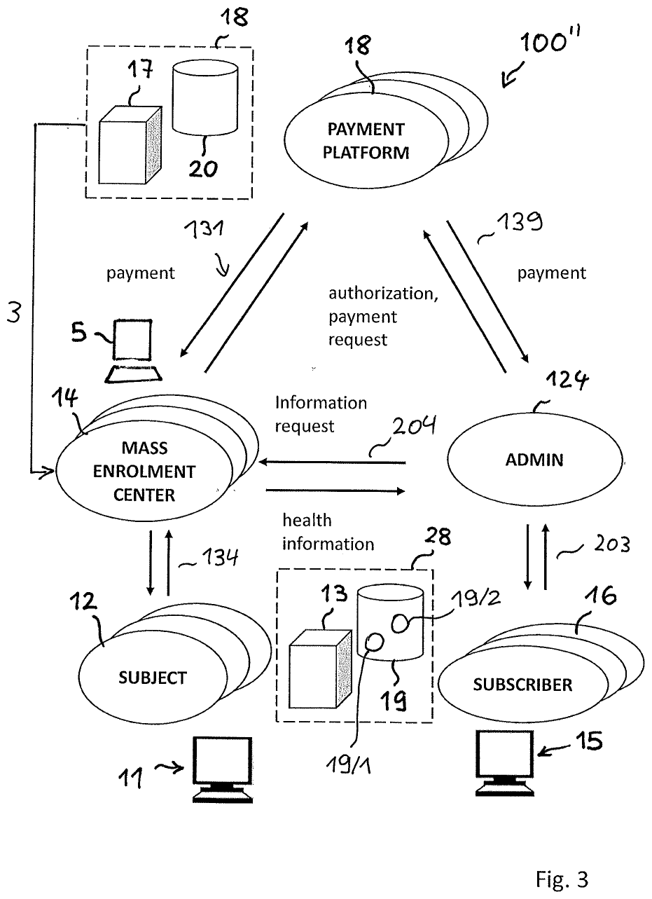

FIG. 3 shows a third embodiment of a medical information exchange system,

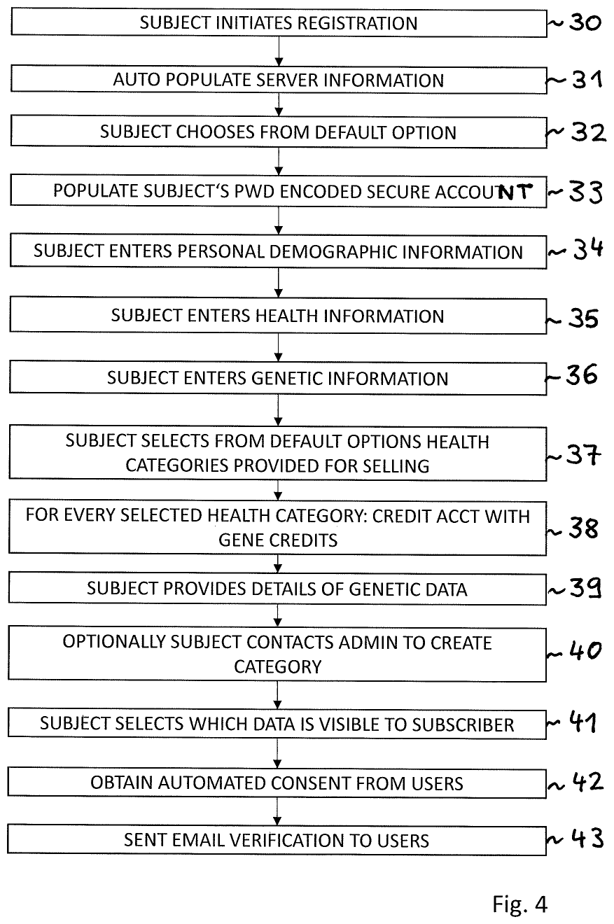

FIG. 4 shows a registration process of a subject in the medical information system,

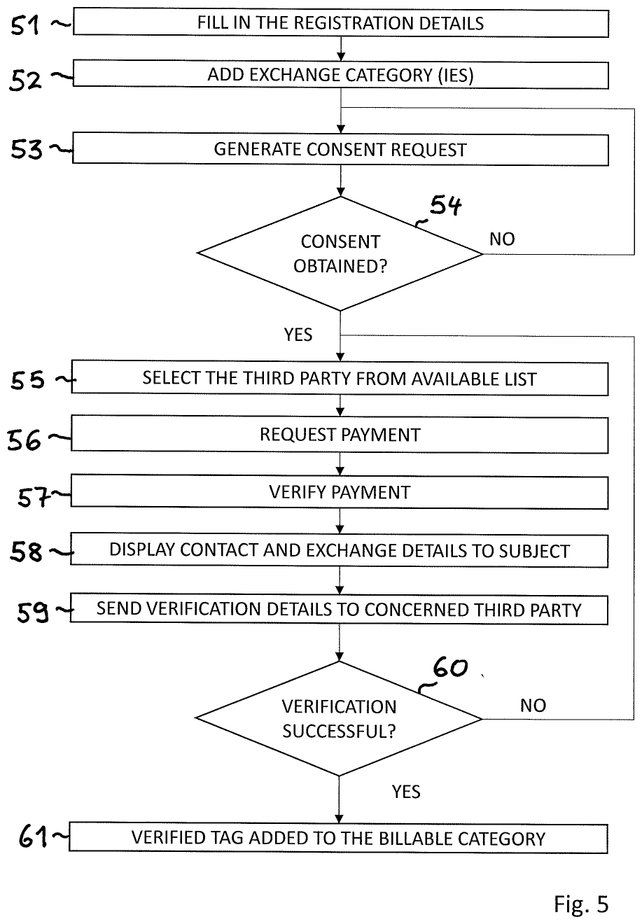

FIG. 5 shows a method of connecting an individual subject with a third party,

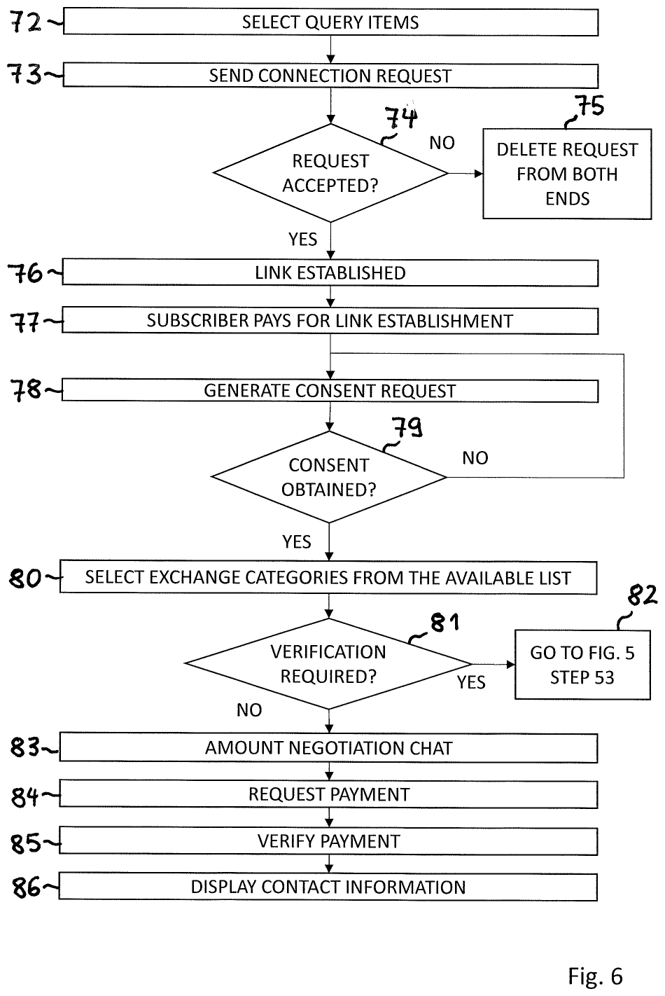

FIG. 6 shows a method of linking a subscriber to a potential subject based on his query search and data indicators shows

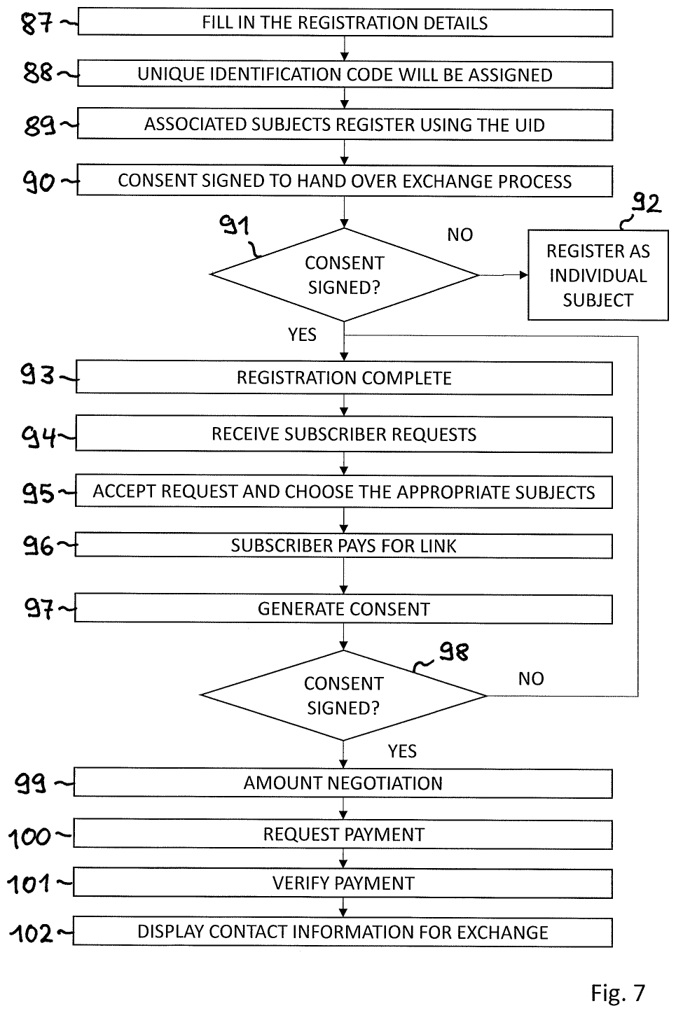

FIG. 7 shows a method of connection between subjects and mass enrolment centers (MEC) and further on the exchange between subscriber and MEC,

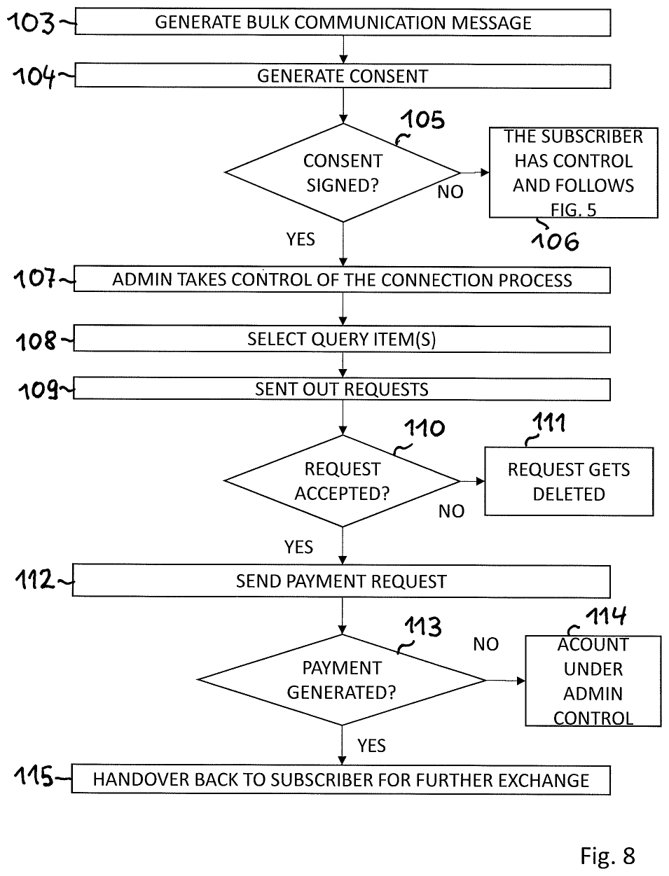

FIG. 8 illustrates a method of handling a bulk communication request between a subscriber and an administrator,

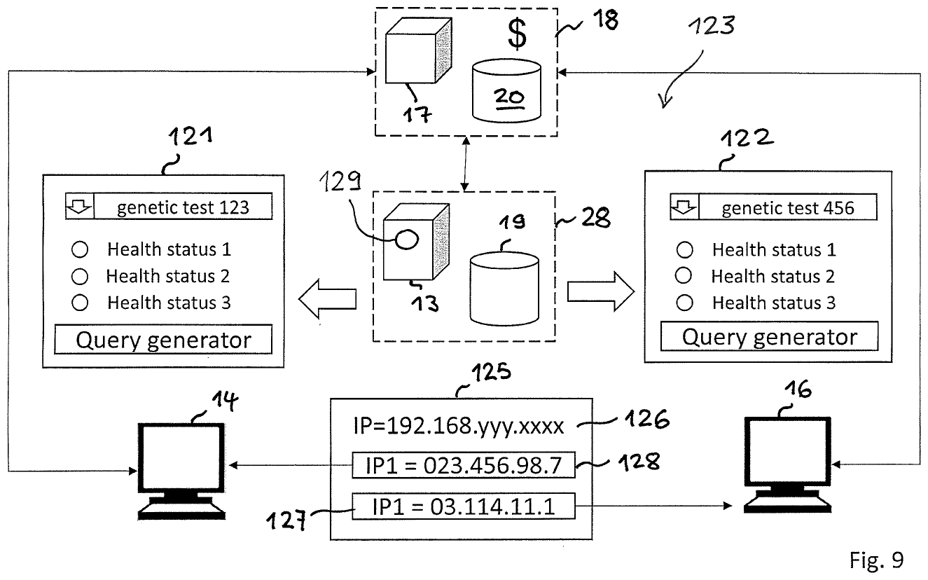

FIG. 9 illustrates a configuration and a method for establishing a safe communication between to parties of the health data exchange system, and

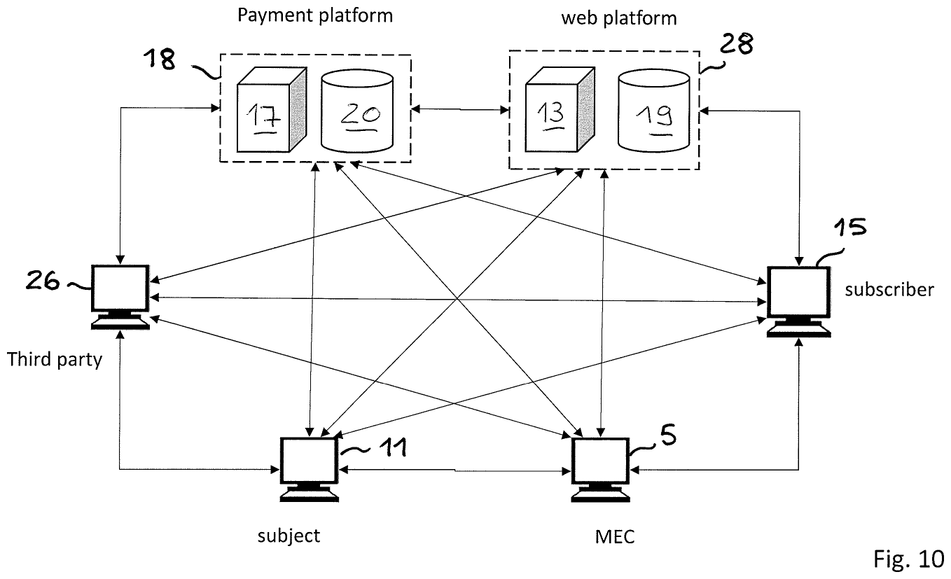

FIG. 10 shows a communication between computer devices of communication partners shown in FIGS. 1 to 3,

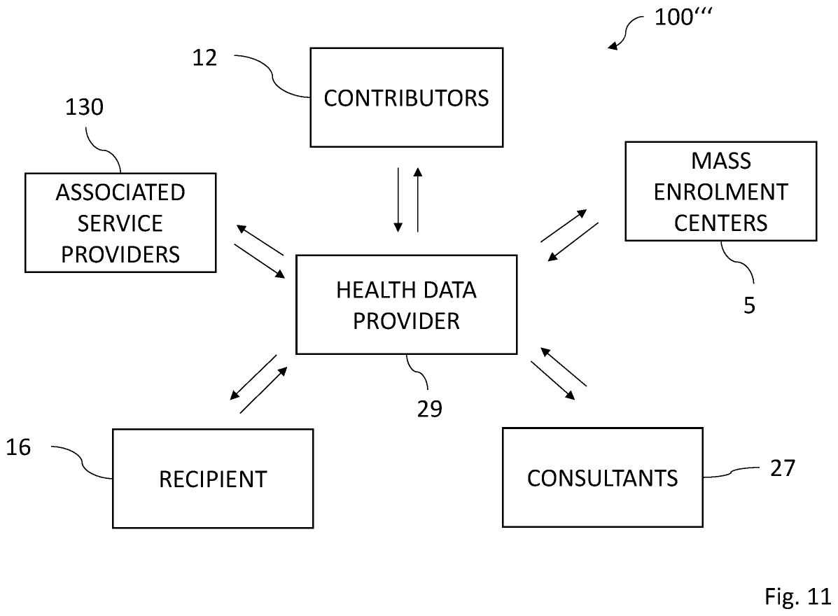

FIG. 11 shows a further embodiment of a health information system,

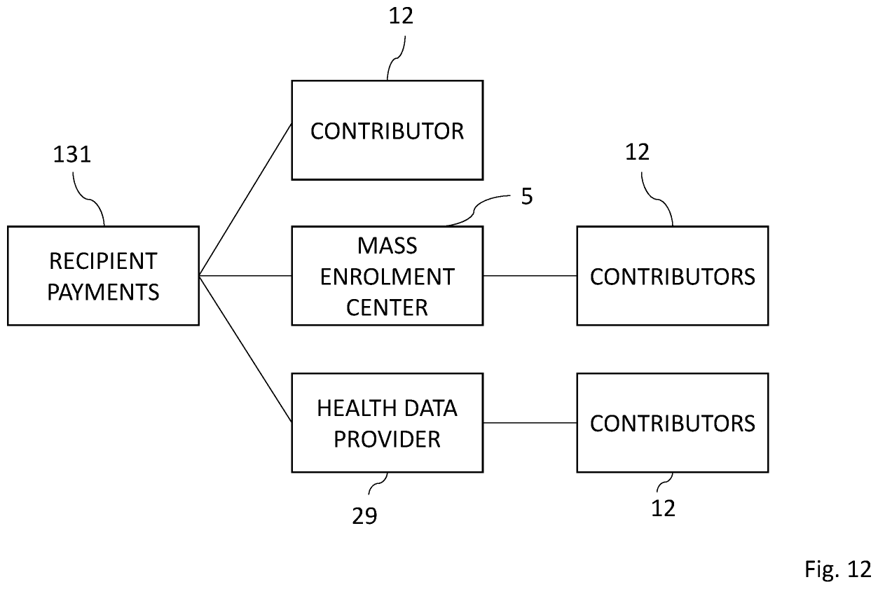

FIG. 12 shows a flow of a recipient payment,

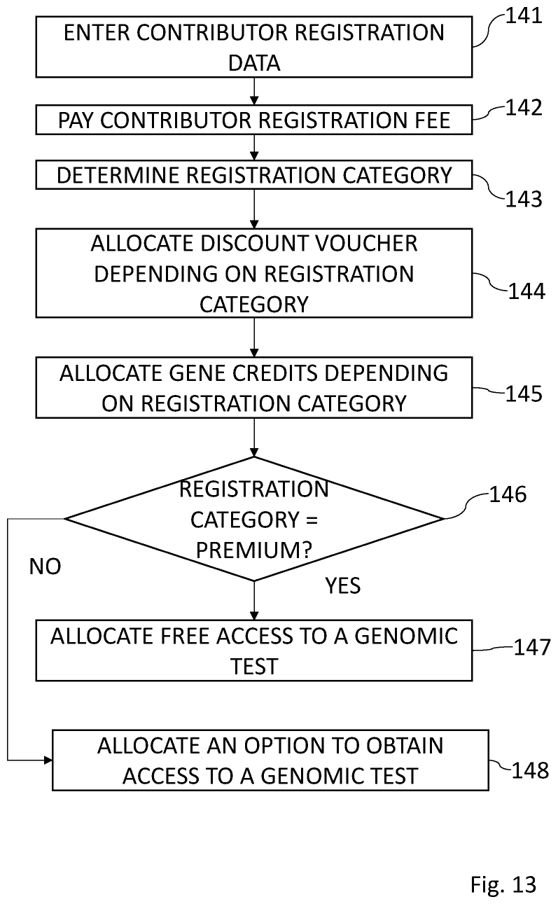

FIG. 13 shows a contributor registration,

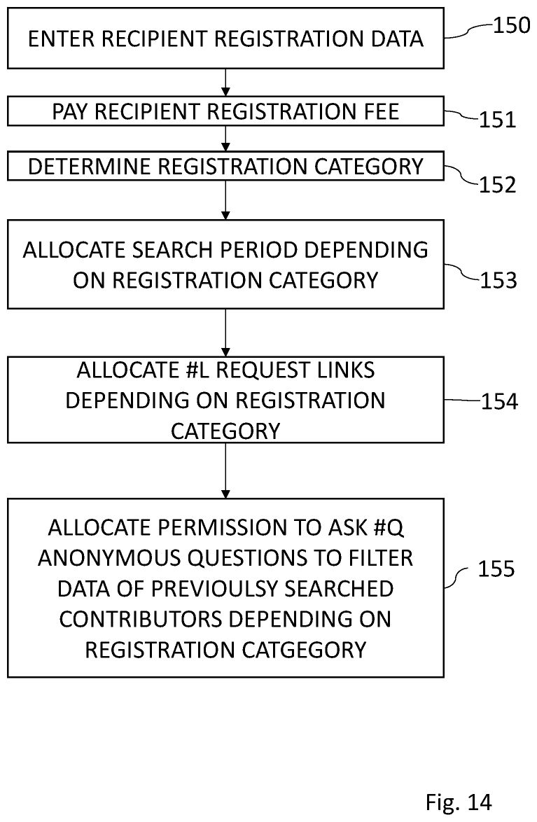

FIG. 14 shows a recipient registration,

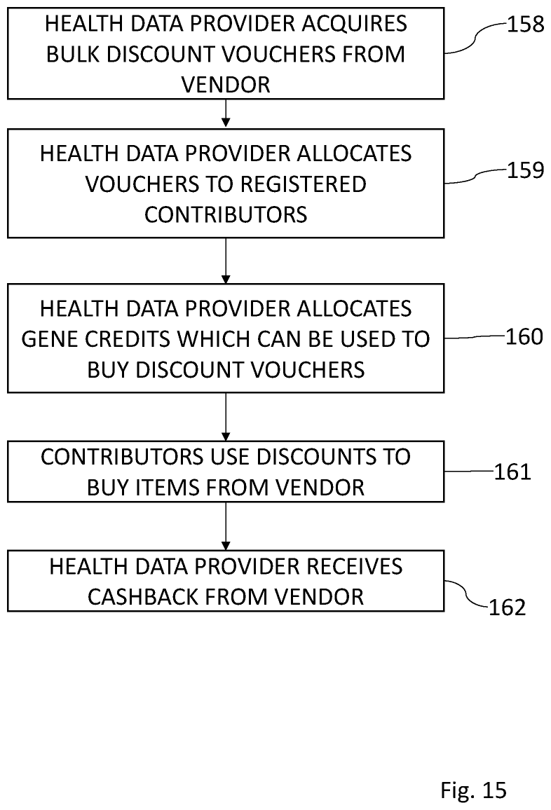

FIG. 15 shows a vendor commission program,

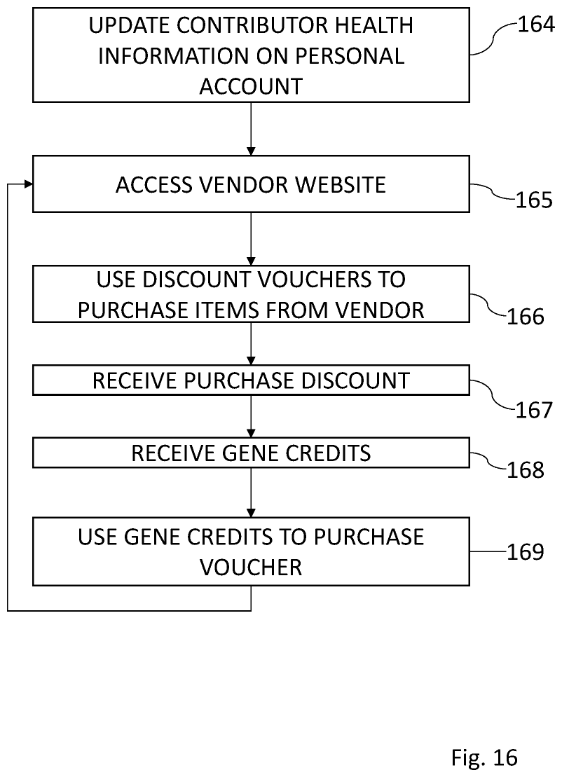

FIG. 16 shows a product purchase and a cashback flow,

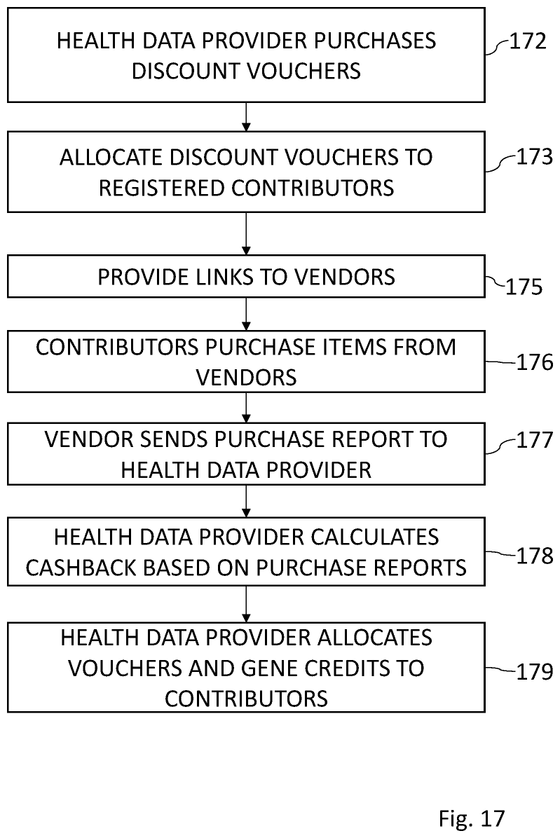

FIG. 17 shows an allocation of discount vouchers to contributors,

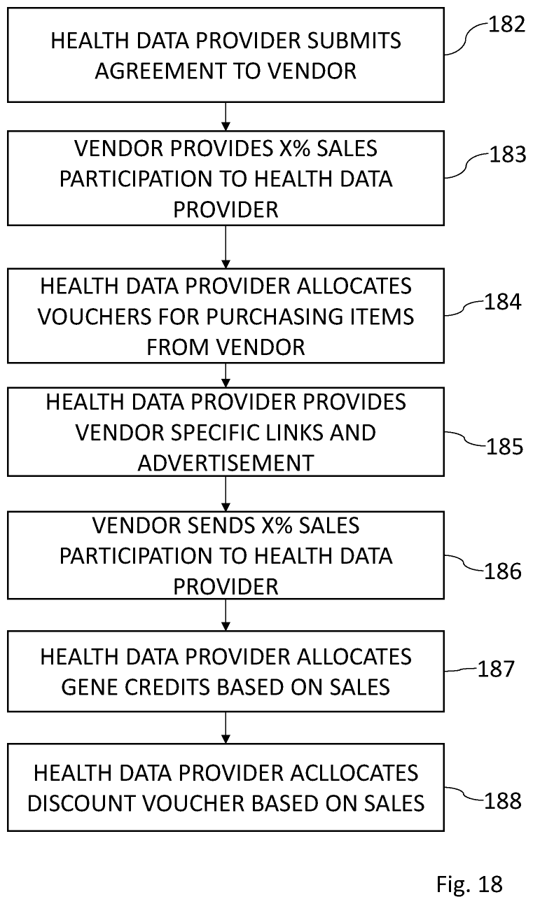

FIG. 18 shows transactions following an establishment of an agreement between health data provider and vendor, and

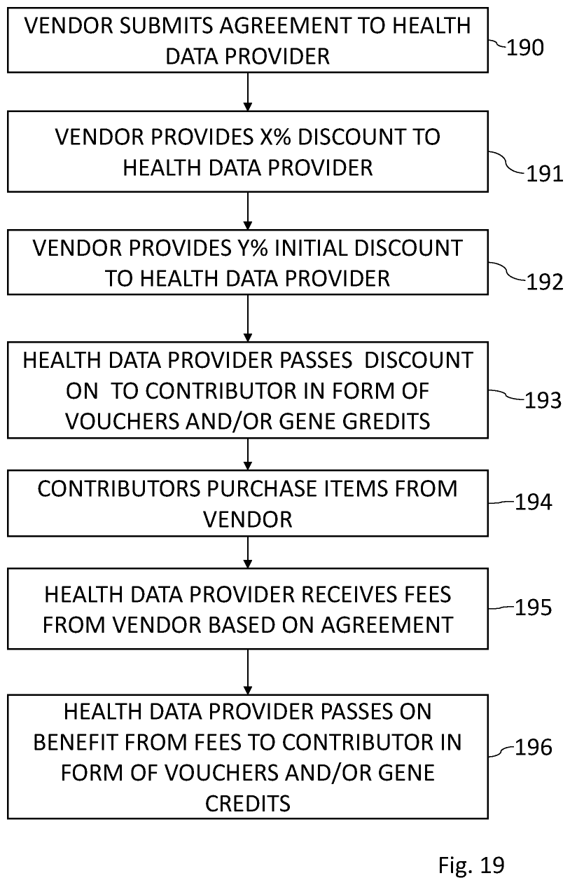

FIG. 19 shows further transactions between health data provider and vendor.

In the following description, details are provided to describe the embodiments of the present specification. It shall be apparent to one skilled in the art, however, that the embodiments may be practised without such details. Although the above description contains much specificity, these should not be construed as limiting the scope of the embodiments but merely providing illustration of the foreseeable embodiments. Especially the above stated advantages of the embodiments should not be construed as limiting the scope of the embodiments but merely to explain possible achievements if the described embodiments are put into practise. Thus, the scope of the embodiments should be determined by the claims and their equivalents, rather than by the examples given.

The following FIGS. 1 to 8 explain a communication between individuals or communication partners which play a role in the transmission of medically relevant information. The FIGS. 9 and 10 illustrate the operation of the communication arrangement used in the health information exchange.

In a general sense, the present specification provides an arrangement for the secret and secure communication by providing a number of transmitting and receiving encryption devices that are set up in a particular manner.

The FIGS. 1 to 3 show three embodiments of an anonymous medical condition information and monetary exchange system, which are also referred to as “medical information exchange systems”, 100, 100′, 100″. The features of these embodiments can also be combined with each other.

In FIGS. 1 to 3, communication arrows are shown between the various communication parties, such as subject, subscriber, administrator, third party, payment platform, and web platform. The communication arrows refer to the communication or, in other words, the exchange of data messages between computers or devices of the communication parties.

The arrows represent communication channels 131-139, 203, 204. The communication channels can be encrypted communication channels, and in particular, the communication channel 135 between the mass enrolment center 14 and the subscriber 16 can be an encrypted communication channel 135.

Furthermore, a payment signal 3 from a payment provider server device 18 to a web platform server device 18 is indicated by an arrow 3 in FIG. 2.

For simplicity, the communication between the respective computers is illustrated in a further FIG. 10. When the specification below refers to a communication between communication parties, this refers to a communication between computers of the communication parties.

Furthermore, the mass enrolment center 14 also refers to a mass enrolment center computing device 14, the subscriber 16 also refers to a subscriber computing device 16, and the payment platform 18 also refers to a payment provider server device 18. For the purpose of showing the payment provider server device 18 and platform 20, the payment platform 18 is also shown separately in FIGS. 1-3 by a dashed line 18.

Specifically, in the following, a communication of the subject 12 with the payment platform 18 refers to a communication between the computer 11 of the subject 12 and the server 17 of the payment platform 18, a communication between the subject 12 and the third party 27 refers to a communication between the computer 11 of the subject 12 and the computer 26 of the third party, a communication between the third party 27 and the web platform 28 refers to a communication between the computer 26 of the third party and the server 13 of the web platform, a communication between the subject 12 and the payment platform 18 refers to a communication between the computer 11 of the subject and the server 17 of the payment platform, and a communication between the subscriber 16 and the payment platform refers to a communication between the computer 15 of the subscriber and the server 17 of the payment platform 18.

Furthermore, a communication between the subject 12 and the mass enrolment center 14 shown in FIG. 2 refers to a communication between the computer 11 of the subject and the computer 5 of the mass enrolment center 14, a communication between the mass enrolment center 14 and the payment platform refers to a communication between the server 13 of the mass enrolment center 14 and the server 17 of the payment platform 18.

Furthermore, a communication between the administrator 124 shown in FIG. 3 and the mass enrolment center 14 refers to a communication between the server 13 of the web platform 28 and the computer 5 of the mass enrolment center, a communication between the administrator 124 and the payment platform 18 refers to a communication between the server 13 of the web platform 28 and the server 17 of the payment platform 18, and a communication between the administrator 124 and the subscriber 16 refers to a communication between the server 13 of the web platform 28 and the computer 15 of the subscriber 16.

The communication between the devices takes place via a communication channel. A communication channel comprises the software infrastructure provided to enable the communication. For example, a communication channel for the communication between communication parties can be provided by the web platform 28. In a broader sense, a communication channel can also refer to the physical infrastructure provided for the communication, such as the hardware.

By way of example, the payment platform 18 can be provided by an internet service such as paypal or other credit or debit card payment services. The payment platform 18 can provide various modes of payment. In particular, the payment platform can provide gene credits 119, which are symbolized by a “$” sign in FIG. 1, and which are explained in more detail further below.

In the following, the user devices of the below mentioned communication partners, such as the user devices of the subject, the subscriber, the mass enrolment center and of the third party are also referred to as “computing devices”. The server of the payment platform and of the web platform are also referred to as “server devices”.

FIG. 1 shows a medical information exchange system 100. The medical information exchange system 100 comprises a first user device 11 of a subject 12, a second user device 15 of a subscriber 16, a third user device 26 of a third party 27, a first server device 17 of a payment platform 18 and a second server device 13 of a web platform 28, which provides services for the users 12, 16, 27 of the web platform 28.

The web platform 28 has an administrator, which is not shown in FIGS. 1 and 2, but which is shown in FIG. 3. The functions provided by the administrator can also be provided by an administrator program with preconfigured settings.

In the example of FIG. 1, the subject 12 and the subscriber 16 are represented by individuals which operate the respective user devices 11 and 15. The third party 27 provides the functions of verification and counselling that allows a large number of subjects 12 to get their health data verified or get counselling for a specific health situation.

In further embodiments, the subject 12 and/or the subscriber 16 comprise an autonomous program running on a user device 11, 15 or on the server 13 which performs its task according to predefined settings.

The respective server computers 17, 13 of the web platform 28 and of the payment platform 18 of FIG. 1 comprise computer memory on which programs and data are provided for handling tasks of the users 16, 12, 65 and of the payment platform 18, and of the web platform 28 respectively.

The server computer 13 of the web platform 28 comprise, among others, a database 19 for storing, among others, health related data 19/1 provided by the subject 12 and identification information 19/2 or identification data 19/2 of the subject 12, third party 27 and of the subscriber 16. Furthermore, the database 19 may also contain the amount and type of data previously requested by the subscriber 16 for various purposes, for example for automatically providing an offer to the subscriber 16. Similarly, the server 17 of the payment platform 18 comprises a database 20 for storing payment information.

In the following, an operation of the health information system 100 is explained by one of many possible usage scenarios. During operation, the subscriber 16 requests a pre-defined health information from the subject 12 by sending a request message 8 to the subject 12. The predefined health information can be selected from a graphical user interface (GUI) which is provided by the web platform 28. For example, the GUI can be generated dynamically using a GUI description language and based on user profile and other data stored in the database 19. By way of example, the GUI can be provided through a web application frame work such as Ruby on Rails or TurboGears, among others.

For example, in one embodiment the GUI includes an anonymous information exchange program with pre-defined selectable information which is presented to the subscriber once the subscriber has paid a contact fee to the payment platform 18.

During the course of communication, if the subscriber 16 wants the data provided by subject 12 to be verified, the subject 12 sends a verification request 9 to the third party 27.

In general, the communication between subscriber 16 and subject 12 can involve more steps than the abovementioned communication steps, for example steps for obtaining a consent of the subject or for successively determining whether the provided information of the subject 12 is relevant for the subscriber 16.

Furthermore, the communication between subscriber 16 and the subject 12 can be handled automatically with message queues. The message queues may be implemented by an application which runs on the server 13. The three parties 12, 16, 27 are prevented from viewing personal identification information which allows them to communicate outside the server 13 until the final payment is made to the payment platform 18.

Once the payment is approved by payment platform 18, the identification information is visible to the respective parties, after which they can communicate outside the server 13 for exchange of information.

In an embodiment which comprises a health provider, and which is also explained further below with respect to FIG. 2, a health provider forwards an information request message to the subject 12. If the subject 12 decides to release health related information and sends back a response message to the health data provider, which forwards the response message to the subscriber 16.

In general, the communication between subscriber 16 and a subject 12 can involve more steps than the abovementioned communication steps, for example steps for obtaining a consent of the subject or for successively determining whether the provided information of the subject 12 is relevant for the subscriber 16.

Furthermore, the communication between subscriber 16 and subject 12 can be handled automatically with message queues. The message queues may be implemented by an application which runs on the server 13 of web platform 28 or they may also be implemented using functionality provided by social media platforms. By using message queues, the messages can be exchanged when the respective party to the automated communication is responsive.

The server 13 is of the web platform 28 is used as a messaging platform to exchange messages between the subscriber 16 and the subject 12. In other words, the web platform 28 as an intermediate for a data exchange between the subscriber 16 and the subject 12. Thereby, the web platform 28 has control over the type of information which is exchanged between the two parties 12, 16. According to one embodiment, the web platform 28 does not provide personal identification data that would allow the subscriber 16 or the subject 12 to identify the respective other party 12, 16.

Furthermore, the web platform 28 allows only the exchange of pre-determined information—and not freely generated messages by the subscriber 16 or the subject 12—between the two parties 12, 16. The pre-defined information can in particular refer to predetermined messages. As a consequence, the two parties 12, 16 are prevented from exchanging identification information with each other which would allow them to contact each other without using the health data provider 14 and without making any payment to the health data provider 14. This applies to all embodiments of the specification.

The subject 12 makes payment for using the web platform server 13 to offer the subject's 12 health information to subscribers 16 by way of sending a payment message to the payment platform 18, which acknowledges the payment by sending an acknowledgement message. The payment platform 18 then informs the web platform 28 by sending a notification message to the health data provider 14.

As a further option, the subject 12 can make the payment for the use of the web platform 28 as a deposit which is reimbursed partially or in full when data of a predefined amount and quality is delivered to a subscriber.

In a simple embodiment, a monetary value of the data provided by the subjects 12 is estimated based on the content of a data input interface which is filled out by the subject. For example, an evaluation can be based on stored standard value estimates for providing information pertaining to certain types of genetic defects. Furthermore, the value of the provided information can be dynamically estimated based on the monitored demand for that type of information. Moreover, the web platform 28 can provide mechanisms to estimate a value of the data of the subjects 12 based on input data provided by a third party, such as experts or specifically trained evaluation personnel.

FIG. 2 shows a further embodiment of the health information exchange platform system 100′. The same or similar parts are indicated by the same or similar reference numerals.

In addition to the features shown in FIG. 1, the embodiment of FIG. 2 comprises a mass enrolment center 14 that acts as an intermediary between subject 12 and subscriber 16, which uses the same server 13 and database 19 as shown in the previous FIG. 1. By way of example, the mass enrolment center can be provided by a health data provider, such as a hospital. The mass enrolment center 14 has a computer or computer system 5, which carries out the exchange of data messages shown in the Figures.

The subject 12 has an option to associate with the mass enrolment center 14 by accepting a consent handing over connection and exchange process through server 13 to the mass enrolment center 14. During operation, the subscriber 16 requests a pre-defined health information from the mass enrolment center 14 by sending a request message 8 to the mass enrolment center 14.

By way of example, the predefined health information can be selected from a graphical user interface (GUI) which is provided by the mass enrolment center 14. For example, the GUI can be generated dynamically using a GUI description language and based on user profile and other data stored in the database 19.

The mass enrolment center 14 then selects from among the associated subjects 12 according to the profile sent by subscriber 16. According to one mode of operation, the mass enrolment center 14 does not provide personal identification data that would allow the subscriber 16 or the subject 12 to identify the respective other party 12, 16.

The subscriber 16 pays for the link through the payment platform 18, after which the link process takes place on server 13. For example, in one embodiment the GUI includes an anonymous information exchange program with pre-defined selectable information which is presented to the subscriber once the subscriber has paid a contact fee to the payment platform 18.

In general, the communication between the subscriber 16 and the mass enrolment center 14 can involve more steps than the abovementioned communication steps, for example steps for obtaining a consent of the mass enrolment center 14.

Once the final payment is made through payment platform 18, the contact information is displayed to the subscriber 16 of the mass enrolment center 14 and vice versa, after which they can communicate outside the server 13 for exchange of information. The transfer of money between the mass enrolment center 14 and subject 12 also takes place outside server 13 as consented.

According to a further embodiment, the web platform 28 provides a query server which is used as a proxy for the subscriber 16 for managing health data queries. The query server retrieves data from the database 19, which comprises profile information about the type and amount of information the subscribers are interested to obtain and about past queries.

During operation, the query provider receives a requirement profile from the subscriber 16 and uses the requirement profile to generate an automated data exchange between the query provider and subjects 12 or between the query provider and the database 19 of the server 13, which contains health information of subjects 12 that matches with the requirement profile. A data transfer message 7 between the respective subjects and the mass enrolment center 14 is indicated in FIG. 2, which can comprise among others an information request, the corresponding released information and consent data.

FIG. 3 shows a health information exchange platform arrangement 100″ providing bulk communication requests to subscribers 16, which allow the subscribers 16 to get into contact with multiple subjects 12. The same or similar parts are indicated by the same reference numerals. Additionally, the concept of an administrator 124 has been introduced.

In the health information exchange system 100″ of FIG. 3, an administrator 124 is used as a proxy for the subscriber 16 for managing health data queries. The administrator 124 uses the web platform 28 for carrying out his tasks. As mentioned before the tasks of the administrator 124 can also be provided by a program running on the server 13 with preconfigured settings.

During operation, the administrator 124 receives a requirement profile from the subscriber 16 and uses the requirement profile to generate a search for subjects 12 or, alternatively, between the administrator 124 and the database 19 of the server 13, which contains health information of subjects 12 that matches with the requirement profile.

The administrator 124 provides a list of subjects 12 to the subscriber 16 after a connection fee payable through the payment gateway 18. Once the payment is verified the further exchange process between the subject 12 and subscriber 16 will take place as per the process of FIG. 5.

For simplicity, the administrator 124 of the web platform 28 is only shown in FIG. 3 for highlighting the functionality provided by the administrator 124 in the context of the embodiment of FIG. 3. An administrator 124 is not shown in FIGS. 1 and 2 although an administrator 124 is also present in the other embodiments.

In the following, a finance model using gene credits is explained. The account of subject 12 is auto-credited with gene credits 119, through the following mechanisms.
Registration of subjects 12 on website server 13.
Transaction between subject 12 and subscriber 16 on server

A percentage of transaction value is auto-allocated as gene credits 119 to the account of subject 12.
Transaction between the mass enrolment center 14 and subscriber 16 on platform 18. A percentage of the transaction value is auto-allocated as gene credits 119 to subject account.
A percentage of the company's profits is transferred to the account of subject 12 as gene credits 119, wherein “the company” is the company that operates or owns the web platform.

These gene credits 119 amount to a dollar value shall be exchanged and used to provide a free or discounted list of genomic tests 120.

The gene credits 119 translate to cash which shall be pushed back to the subject 12. The gene credits 119 translate at a later date to ownership of the company and shares in the company.

In the following, a finance model based on free tests is explained. This finance model is developed to have a self-sustaining construct, pay for free tests and build a database of whole genome sequence at a global level. It also provides cash benefits to Subjects in addition to potential free tests.

Gene credits 119 are used to provide a default list of genomic tests default list of genomic tests 120 to subject 12 for free or at a subsidized rate. The list of available tests is determined by the administrator 124. The list of tests includes but is not limited to whole genome sequencing. The company that operates the web platform 28 preferentially provides this default list of tests free to subjects 12. However, the subject 12 may also purchase gene credits to purchase the listed test 120.

Preferentially, the operator of the web platform 28 procures the test from its subsidiary company and or its affiliates although it may also be provided by the web platform 28. The subsidiary company shall organize testing and logistics. The free test information and data shall be posted on subject account 12. This shall be pre consented at the time of registration as a precondition to provide free test.

According to one embodiment the web platform 28 provides free tests including but not limited to whole genome sequencing (WGS) of an individual. WGS, full genome sequencing, complete genome sequencing, or entire genome sequencing) is the process of determining the complete DNA sequence of an organism's genome at a single time. This entails sequencing all of an organism's chromosomal DNA as well as DNA contained in the mitochondria and, for plants, in the chloroplast. In practice, genome sequences that are nearly complete are also called whole genome sequences.

Sequencing can generate a large amount of data. This data shall be available for research and for science through server 13. With improvements in artificial intelligence and informatics this data, if reanalysed at a later date in time, can provide new medical and health information to the subject. The company shall provide this service of data reanalysis of free tests for a fee at a subsequent period in time.

Furthermore, the health data provider can provide reanalysis packages for cancer predisposition or ancestry testing.

According to a further example, the health data provider provides free personalized genomic tests for contributors every month and subsidizes exome and tumor tests with a test provider. The test provider executes the tests. Thereby, the health provider can expand its test menu and increase and expand its geographic presence. The test provider sends the test data back to the health data provider for automatic update on the web platform. This enhances the data basis of the health provider which can be searched by the recipients, which enables the health data provider to provide more free tests and gene credits to the contributors.

In the following, a further finance model is explained, which is based on a genomic stock exchange. This is a concept based on the fact that the administrator 24 is able to create digital gene assets on server 13 and ascribe a fixed monetary value to each.

The platform 13 allows for a link between subject 12 and subscriber 16 to perform sale and purchase activity. Therefore, there is a volume of transaction and number of transactions linked to each GSC. This allows for development of a new platform called genomic stock exchange. This new platform can be realized on the web platform 28, the payment platform 18 or a separate provider not shown in FIGS. 1-3.

The web platform 28 allows for Trade of genetic stock exchange (GSC) shares as commodities. A trade transaction on platform 28 generates a revenue. A percentage of this revenue shall be passed back to the account of subject 12 as gene credits 120. Gene credits are used to provide free test and cash benefits to subjects.

FIGS. 4 to 8 show processes carried out by the medical information exchange system 100, 100′, 100″. In particular, FIGS. 4 to 8 illustrate the processes of registration of a subject, carrying out a communication between a subject and a third party, carrying out a communication between a subscriber and a subject, FIG. 4 shows a workflow for a registration process. First, the subject 12 reviews and sees the website hosted on server 13 on his user devices 11.

In a step 30, the subject 12 chooses to register on the server 13. In a step 31, all the information on the server 13 is auto populated and, in a step 32, the subject 12 chooses from default options predefined on the server 13 to populate the password encoded secure account of subject 12 which is hosted on server 13 in a step 33. There is automation to allow ease of registration.

While registering, the subject 12 provides personal demographic information including age and gender apart from other information which may be useful for the subscriber 16 in a step 34. To simplify registration, the subject 12 inputs demographic data in step 34. Then subject 12 signs on which digital gene assets he is willing to share, and then creates an account using e-mail verification, including agreeing to consent. In case of payment he pays at the time of creating an account. The account is created first, and the health and genetic information can then be updated at the same time or later by the subject after the simple registration.

The subject 12 then provides health information which is information related to health and disease in a step 35. The subject 12 may be healthy, apparently healthy, or diseased. Subjects with any kind of available health information are invited to register on the website platform hosted on server 13.

The subject 12 then proceeds to provide available genetic information which he has readily available and is willing to share on the platform 13 and with subscribers 16 in a step 36. The subject 12 then selects from default options which exchanges category he is willing to sell in a step 37. The predefined options may comprise for example willing to exchange specific genetic data, register for research trials or share a specimen. For every digital gene asset that the subject 12 is willing to opt for, the account is credited with gene credits in a step 38.

The subject 12 then provides details about the genetic data which is stored internally on the server 13 and which is linked to a rate fixed by the administrator 24 of the web platform 28 in a step 39.

All digital gene assets and data category are linked to default recommended rates fixed by the administrator 24. Only the administrator 24 is authorized to create new digital gene assets.

There is an option for mass enrolment center 14 to negotiate a rate with the administrator 24. The difference in rates is based on geographical and finance concerns of the subject 12 or mass enrolment center 14. As a further option, if data with Subject 12 does not fall into the provided digital gene assets, the subject 12 may contact administrator 24 to create a new category in an optional step 40.

To increase the data on the subject's account 12 which is used for profiling and search by subscriber 16 or mass enrolment center 14 or administrator 24, free text description of health state is permitted, though limited by number of characters used. In another unique workflow feature, the website on server platform 13 allows the subject 12 to opt for which of the data shall be made visible to subscriber 16 in a step 41. Once registration of the users 16, 12 or 14 is complete on server 13, an email verification is sent to verify these users in a step 43.

The server 17 allows for two kinds of subjects to create an account: standard subjects 12 and premium subjects 12′. A premium subject 12′ pays fees to the web platform 28 via the server 13 of the web platform 28 while registering using payment platform 18. A premium subject 12′ gets Free gene credits while registering. No real time data is posted on the server 13. Only information about the availability and willingness to share is posted on server 13.

An automated consent is obtained during registration from all the users 16, 12 and 14 agreeing to terms and conditions in a step 42.

FIG. 5 shows a workflow of connection between an individual subject 12 and a third party 27. More specifically, FIG. 5 shows a method of connecting an individual subject with a third party, such as a medical examiner, for the process of verification of reports, raw data, result or test.

In a first step 51, the subject 12 registers on the website 28 using the subject registration form, entering his personal data as well as health information.

After the registration, in a step 52, the subject adds the billable categories/data for the exchange process. For the subject 12 to get the data verified, a consent is generated at step 53 before the start of the verification process.

In a decision step 54 it is decided whether a consent has been obtained. If the consent is obtained in the decision step 54, a third party is selected in a step 55 for the verification process from a list of all the available third parties 27. The subject 12 selects the third party 27 through a filtered list of third parties based on country, state and city. The he can select any suitable third party 27 and proceed to get counselling or verification.

If it is decided in step 54 that a consent could not be obtained, the process loops back to step 53 to generate a new consent request.

In a further step 56, a payment request for a fixed amount is generated. Once the payment is successfully verified in a step 57, the subject 12 receives the contact information of the third party to send in the data to be verified in a step 58.

A verification request is displayed on the third-party portal in a step 59. The actual handoff of data occurs through email or physically outside the web platform 28, or, in other words, outside the web application provided by the web platform 28.

Once verification is complete in step 60, the third party assigns the status of “verified” or “failed” to the particular billable category on the subject side in step 61.

According to a further embodiment, a third-party verification is carried out without selection by a third party or a recipient. Instead, the system or the health data provider web site autogenerates or auto-selects a third-party verifier with whom a link is created to verify data. Thereby a selection bias can be avoided.

FIG. 6 shows a workflow of connection between an individual subject 12 and a subscriber 16. Specifically, FIG. 6 explains the method of linking a subscriber 12 to a subject 12 matching the requirements of the subscriber 12 and based on the query search and data indicators of the subject 12.

In a first step 72, a set of query items is selected. In step 73 a request is sent to the potential subjects 12. Once the request is accepted in step 74, a link is established between the potential subjects and the subscriber in step 76, respectively. Otherwise, the request is deleted from both ends in step 75.

The Subscriber pays a fixed amount for link setup in step 77, after which consent is generated in step 78. In a decision step 79, it is decided whether a signed consent has been obtained from the subject.

If it is decided in step 79 that a signed consent has been obtained, the subscriber has an option to choose from available reports (billable categories) in step 80. If it is decided, in a decision step 81, that a verification is required, step 82 is followed wherein the subject gets their data verified by the third party by branching to step 53 of FIG. 5 and completing step 53.

Once the data to be exchanged between the parties is agreed upon, an automated anonymous information exchange negotiation feature is available in step 83 for them to finalize upon the price. During an amount negotiation the subject 12 either agrees to a recommended rate or disagrees. If the subject 12 does not agree, the subject is the first person allowed to start a negotiation. He is allowed to choose a value within a fixed percentage up or down from the recommended rate, such as plus or minus 10%. The subscriber 16 either agrees or recommends a different rate, again ranging 10% up or down from the recommended rate. The subjects 12 then accepts or declines. Only this much of negotiation is allowed.

Payment is requested in step 84 and is made by the subscriber 16 through a secure payment gateway in step 85, after which contact information of the subject 12 is provided to the subscriber and the contact information of the subscriber 16 is provided to the subject 12 in step 86. The actual exchange of data happens via mail or physical hand-off outside the web platform 28.

FIG. 7 shows the establishment of a connection between subjects 12 and mass enrolment centers (MEC) 14 and furthermore an information exchange between subscriber 16 and MEC 14. The establishment of the connection, which comprises steps 87-93 of FIG. 7, is described by a first workflow below and the information exchange, which comprises steps 94-102 of FIG. 7, is explained by a second workflow below.

FIG. 7 shows a work flow of connection between subjects 12 and mass enrolment centers (MEC) 14 or a health data provider.

The MEC will register on the website through the MEC registration form in step 87. On registration, the MEC 14 will be provided with a unique identification code (UID) in step 88. This UID will be used to link the subjects 12 with the MEC 14.

While registering, in step 89 the subject 12 will enter the UID associated with MEC and will be provided with an option that gives consent to the MEC for handling all their exchange processes and receiving payments on their behalf and that the provider of the web platform 28 will not be liable for distribution of money between the MEC and subjects in step 90.

A two-tier security is inbuilt. At the first level, the identities of the subjects 12 remain hidden and can be identified by the administrator only through the unique identification code (UID). At a second level, a security password is provided to the administrator 124, who can then identify personal information, which is accessible by the username and password of the user. This access is provided to the administrator 124 in order to enable the administrator to access transaction data such as payment transfers or gene credit cashbacks. As a further security feature, an automatic logoff timer is provided for the administrator account.

Among others, the health information system 100, 100′, 100″ provides the following security features:

The health information system provides an SSL-protected connection between the communication partners and between a communication partner and the web site. Thereby connections to other sites and the server linked to the website provided by the web platform 28 can be secured.

To that end, the web server 13 provides an implementation of SSL certificate to make the website respond to https (hypertext transfer protocol—secured) queries, for example by purchasing and implementing a third-party SSL certificate for the website from a reputed certification authority. Furthermore, a testing of the strength of the certificate is carried out from the service provider's end as well as the developer's end. The SSL communication is an example of an encrypted communication channel.

Moreover, the health information system 100, 100′, 100″ provides an anti-forgery token implementation. This implementation includes among others the following features: prevention of cross site request forgery attacks, and thereby preventing forms used for login ID and passwords, throughout the website from being forged onto another dummy website, not displaying passwords in clear text. Including the anti-forgery token into the framework of the website.

Furthermore, the health information system 100, 100′, 100″ provides an advanced encryption standard, preferentially a 128 bit AES, to encrypt the data throughout the website. By using this encryption, the transferred data can be encrypted on the redirected websites as well. In particular, the AES 128 bit encryption can be implemented and tested through the SSL certificate implementation.

Moreover, the health information system 100, 100′, 100″ provides an exclusive end-to-end communication between the subscriber 16 and the subject 12 will be exclusive. In particular, the anonymous information exchange between subscriber 16 and subject 12 can make use of the AES 256 bit encryption.

A further security feature provided by the web site is payment gateway security. In particular, a payment gateway security can be implemented by reputed service providers and it can be implemented on PCI DSS standards (payment card industry data security standard).

Furthermore, the health information system 100, 100′, 100″ can provide periodic assessment of security, which may be carried out in an automated, partially automated or manual way.

According to one scheduling of the security monitoring, the security monitoring of the website takes place every month and the assessment will comprises the following elements.
Vulnerability assessment—Determining if there are any web related vulnerabilities on the website.
Penetration testing—Assessment of the risk caused to the website due the vulnerabilities.
Patching—Implementing corrective measures for the vulnerabilities
Testing of the following parameters and their corrective measures, wherein test reports for these tests can be submitted with the assessment monthly:
Storage of an unknown code in the website framework—cross site scripting (XSS)
Unauthorized log in to the website through a dummy website
Cross Site Request Forgery (CSRF)
Strength of the SSL certificate and encryption—Heartbleed vulnerability assessment.
Login through arbitrary SQL query on the website—SQL injection.
Denial of service attacks.
Testing for server errors which can give access to website database.

If it is decided in step 91 that the consent is signed, the subject 12 will only be receiving notifications on the exchange processes happening but will have no control on handling the exchange process. The registration is completed in step 93, when it is detected in decision step 91 that a signed consent has been obtained. Otherwise, a message is sent to the subject for which no consent has been obtained to register as an individual subject in step 92.

On the other hand, the MEC will be signing a consent in step 87, saying that they are appointed on behalf of the subjects 12 and will be receiving payments for the entire exchange process. Furthermore, the consent includes a statement that the provider of the web platform 28 will not be liable for distribution of money between the MEC and subjects 12.

Furthermore, FIG. 7 shows a workflow of connection between MEC and Subscribers.

The MEC has a general profile of the type of subjects 12 associated with them according to which the subscriber 16 can send a connection request to the MEC in step 94. Once the MEC accepts the subscriber request, it sorts through the associated subjects in step 95 according to the subscriber requirements and creates a connection.

The subscriber 16 pays a fixed amount, which depends on the number of subjects 12, for the link to the web platform 28 in step 96 and then a consent is generated in step 97. After it is decided that the consent is successfully signed between the MEC 14 and the subscriber 16 in step 98, an automated anonymous information exchange feature will be provided for amount negotiation between the MEC 14 and the subscriber 16 in step 99.

Once the amount is finalized and payment requested in step 100, the subscriber 16 makes the payment in step 101 and both MEC 14 and the subscriber 16 will be able to view each other's contact information in step 102. At a later stage, the exchange of actual data will be done on a mail or by a physical handoff outside the web platform 28.

FIG. 8 shows a work flow of connection between subscriber and admin. More specifically FIG. 8 illustrates a bulk communication request between the subscriber 16 and administrator 124.

The subscribers 16 have an option to provide search and connection control to the administrator 124 of the website as query provider. The administrator account is preferentially handled by one of the members from the web platform 28.

In step 103, a bulk communication message is generated. A subscriber 16 chooses the administrator of the health information exchange system as the query provider in step 103. Next, a consent is generated in step 104. On deciding that the consent has been signed and accepting the consent in step 105, the administrator 124 takes control for subject search and sending out requests on behalf of them in step 107. The subscriber 13 for that period of time can only view the process on the portal.

If it is decided that the consent has not been signed in step 105, the control is returned to the subscriber 13 and the process loops back to step 51 of FIG. 5. The administrator 124 selects the subjects according to the filters or query items provided by the subscriber in step 108 and sends out connection requests to them in step 109.

Once it is decided that a response is received from the subjects 12 and the request is accepted in step 110, the administrator request a link payment in step 113 and sends the list of subjects 12 to the subscriber 16 after the link payment in step 112. If the request is not accepted in step 110, the request is deleted, and the process ends in step 111. Similarly, if no payment is generated in step 113, the control is returned to the administrator 124 in step 114, and the administrator decides about further steps to be taken.

The control is handed over in step 115 to the subscriber 16 for further process of signing the consent, selecting from the digital gene assets and for making the exchange payment, similar to the individual subject-subscriber connection, as described above with reference to FIG. 5.

FIG. 9 illustrates a method of providing secure information exchange in a health information exchange system 100, 100′, 100″ according to the present specification.

According to this method, the web platform 28 creates a link site with a unique identifier when a communication between two parties is required, for example a communication between a subscriber 16 wanting to obtain health or genetic information from subjects 12 and a mass enrolment center 14. The link site can use website technology, for storing and displaying information, and also communication means, such as messenger technology for exchanging message between the mass enrolment center and the subscriber.

The link site is created as a temporary link site, which implies that after an expiry condition is fulfilled the link site is no longer available or is no longer available for the intended communication for which the link site was created. The temporary link site is thereby made unavailable, which is also referred to as “deleting” the link site. For example, a provided link can point to some static information or to an error message page after deletion of the link site. By providing the link site as a temporary link site, the security can be increased.

Different from generating only a temporary link to information in the masse enrollment center computing device 14, providing a temporary link site enables collection of additional data, such as communication data or pre-determined messages. By doing so, the matching between the subscriber 12 and the subject 16 can be improved.

By way of example, an information about the subject 12 can be provided in the following data structure

subject {
identity information;
subject identifier code;
health data type;
genetic data type;
},

in which “health data type” and “genetic data type” provide generic information about the type of health data or genetic data being provided by the subject, but not the genetic data and/or the health data itself. This “health data type” and “genetic data type” data is stored on the database 19 of the server 13. The identity information is personal data of a subject and it is—unless sent out automatically to a communication partner for concluding a transaction—only accessible to the administrator 124. The other parties, such as the third party 27, the subscriber 16 or the payment system 18 only know the identifier, here referred to as “subject identifier code”.

By way of example, the data structure or part of it can be passed over the net as part of a response to an information request, for example using JSON or an XML query. By only storing and transmitting generic data it can be prevented that any party to the communication gains direct access to the detailed identity information. As a security measure, the actual genetic data and health data is not stored on the server 13 but is only retrieved from the subject 12 and stored on the server 13 once the subject 12 has made a payment, or after the subject 12 has made the payment.

According to one example of releasing the identity information data, the web platform server 13 automatically initiates a transmission of identity information data of a subject 12 after the server 13 of the web platform 28 has received a corresponding payment signal from the server 17 of the payment platform 18 or, in other words, upon reception of the payment signal.

The parties to the communication, for example a subscriber 16 and a mass enrolment center 14, can only exchange information via predefined messages. This is illustrated in FIG. 9 with GUI input masks 121, 122 having GUI input elements. The GUI input masks provide an example of a communication interface 121, 122. Alternatively, the same or similar functionality can also be provided by a command line interface or another standardized exchange of messages. The last two options facilitate automated communication, for example at the end of the mass enrolment center 14, where a party to the communication can be represented by a program, a batch script or the like.

The GUI, the command line interface, and the standardized message exchange provide examples of communication interfaces. Preferentially, the messages that are exchanged using the communication interface are encrypted by one of the abovementioned methods, such as SSL, AES encryption and so forth.

A communication between the two parties is established via a communication infrastructure 123, which includes the software, hardware and data-structures required to establish and maintain the communication.

During runtime the GUI elements of the input masks 121, 122 are filled with the predefined options available from the database 19. By way of example FIG. 9 shows a combo-box with pre-defined elements, an option field with pre-defined options and a button for loading a query generator, which a party to the communication can use to generate a query using predefined elements from the database 19 according to pre-defined rules, which may be stored in the code, the database, in configuration files or elsewhere.

At the beginning of a communication request, a subject 12 checks the identity of the website provided by the web platform 28 and selects a health provider or a mass enrolment center 14 for submitting his or her data.

After that, a program of the web platform 28 and/or the administrator 124 of the web platform 28 invites the subject 12 to pay a fee. The subject 12 provides payment by interchanging communication signals with the payment gateway 18. This process in shown in FIG. 9 by a double arrow.

After receiving the payment from the subject 12, the web platform 28 creates a link site 125 with a unique identifier 126, such as a dedicated IP address 126, and permits communication over the link site with a communication partner, such as a subscriber. In particular, the link site 125 can be created on the server 13 using an available pool of IP addresses which has been obtained by the provider of the web platform 28 and which is indicated in FIG. 9 by the placeholders “yyy.xxxx”.

The temporary link site 125 and the temporary link site identifier 126 are provided stored in a computer memory link location 129, which may be provided in a read and write memory or by a read only memory. The temporary link site 125 comprises the communication interface 121, 122, among others. The communication interface 121, 122 is adapted for the exchange of predetermined messages between the mass enrolment center computing device 14 and the subscriber computing device 16.

Furthermore, the web platform 28 stores the respective IP addresses 127, 128 of the communication partners, which allows to re-establish the communication when it is interrupted and to include the IP addresses into a protocol. The protocol can be used, among others, to trace back a communication partner after the communication is terminated.

After a termination condition is satisfied, the web platform 28 terminates the link site 125, after which the link site 125 is no longer available under the earlier provided IP-address or, as an alternative possibility, the link site 125 is available but it does not display a communication dialog or interface as before but some other information.

Among others, the method shown in FIG. 9 can reduce a delay of providing the health information. This can be advantageous for example if patients die between the diagnosis and the matching between subject 12 and subscriber 16, a large amount of data can be covered, a corruption of the data can be made less likely and a data protection can be improved.

By way of example, a matching of data between the provided data of the subject 12 and the required data of the subscriber 16 can be carried out as follows. The required data of the subscriber 16 is transmitted from the computing device of the subscriber to the web platform 28 in the form of a predetermined message. The server 13 of the web platform 28 determines whether the provided data of a subject 12 or of multiple subjects 12 falls within the scope defined by the predetermined message of the subscriber 16. When this is the case, the server 13 triggers a further action, such as sending a payment request message to the computing device 15 of the subscriber 16. When the server 13 receives a message that the payment has been made, it performs further steps to enable the computing device 15 of the subscriber 16 to establish a communication with the computing device 11 of the subject 16 and to retrieve the required data. Thereby, an automatic verification of a matching of health-related data is provided. Herein, a computing device 11 of a subject 16 can also be represented by a proxy device, such as a computing device 5 of a mass enrolment center 14.

The abovementioned method of establishing a communication between communication partners of FIG. 9 further provides that the administrator 124 is not able to see the connection or communication channel between the subject 12 and the subscriber 16, which increases the degree of protection of personal data.

According to a second level of security, an operator or administrator of the web platform 28 is prevented from seeing the detailed information exchanged between the matching subject 12 and subscriber 16.

In the same way that the mass enrolment center 14 can provide a payment to the payment platform 18 and thereby trigger an action of the web platform 28, other communication partners, such as the subscriber 16 or even the web platform 28 can also provide a payment to the payment platform 18. This is symbolized by double arrows in FIG. 9. For example, the provision of the link site can be made when the mass enrolment center 14 and/or the subscriber 16 have provided a payment to the payment platform 18.

FIG. 10 shows the exchange of data, especially data pertaining to health related and payment related information between the computing devices of the various communication parties. In principle, a data change can take place between any two computing devices of the respective communication parties, which is illustrated by the arrows of FIG. 10.

In order to achieve this, the arrangement for the secret and secure communication provides a number of transmitting and receiving encryption devices that are set up in a particular manner, as described above with reference to FIGS. 1, 2, and 3.

The web platform 28 with the second server device 13 and with the database 19, the first user device 11, the second user device 15, the third user device 26, the payment platform 18 with the first server device 17 and with the payment platform database 20, and the MEC user device 5, form an arrangement for exchanging encrypted information over the a communications network, such as the Internet.

The arrows between the aforementioned devices represent communication links that transmit data and signals from one device to another device. A signal can also take the form of a data message. In particular, the signal can be an electric signal, or a signal in the form of an electromagnetic wave which carries the data.

In other words, the participants to the exchange of personal medical and financial data do not communicate directly with each other, but they communicate by providing inputs to the aforementioned devices or by reading outputs from these devices.

Some of these communications are automatic, such as the emission of a payment signal before establishment of a matching or the transmission of personal identity information upon matching.

Some of these communications can only be provided upon establishment of a link site that has been created by the web platform.

Some of the communications are based on a number of predetermined messages, such as the negotiation between the first user device 11 and the second user device 15.

None of the communications provides individually generated messages, and all communications can only take place between the pre-determined communication partners.

FIG. 11 shows a further embodiment of a health information exchange system 100′″ in which a health data provider 29 is connected to mass enrolment centers 5, consultants 27, recipients 16, associated service providers 130 or partners 130, and contributors 12 over respective communication links in a star topology. The star topology implies that communications between the parties 5, 12, 16, 27, 130 are directed over the health data provider 29, which functions as a switching center or communication broker between the respective communication partners.

The associated service providers 130 provide goods and/or services for the benefit of the other parties 5, 12, 16, 27 via the health data provider 29. For example, they can be vendors of products, insurance providers, tour operators, providers of medical tests and so forth.

In the health information exchange systems 100, 100′, 100″ of FIGS. 1-3, the health data provider 29 is represented by the web platform 28, which comprises the health provider database 19 and the first server 13. The contributors 12 of FIG. 11 correspond to the subjects 12, the recipients 16 of FIG. 11 correspond to the subscribers 16 and the consultants 27 of FIG. 11 correspond to the third parties 27.

FIG. 12 illustrates a flow of direct payment 131 from a recipient to a contributor 12, to a mass enrolment center 5, to a health data provider 29. Furthermore, FIG. 12 shows a flow of indirect payment 131 from a recipient to a contributor 12 via a mass enrolment center 5, and from a recipient to a contributor 12 via the health data provider 29.

FIG. 13 illustrates a registration of a contributor at the health data provider 29 and a mechanism to provide benefits to the contributor.

In a first step 141, a contributor enters registration data, such as personal profile data, contact information and health information. The contributor is able to update the health information at a later time after registration.

In a further step 142, the contributor pays a contributor registration fee, which is generally a small fee such as 5 USD, and which varies according to an intended subscription category. A fixed percentage of the registration fee, such as 10%, is automatically forwarded to the partners of the health data provider.

A subscription category is determined in a next step 143. According to one alternative, the subscription category is chosen depending on the paid fee. According to another alternative, the contributor first chooses the subscription category and/or the sum corresponding to the subscription category and registration proceeds only when the amount required for the subscription category has been paid.

In a next step 144, an amount of discount vouchers is allocated to the contributor according to the subscription category. In a further step 145, an amount of gene credits is allocated according to the subscription category. After registration, a contributor can purchase additional gene credits and the conversion rate into gene credits is dependent on the subscription category.

If the subscription category is determined as a “premium category” in a decision step 146, a free access to a genomic test is allocated to the contributor in step 147. Else, an option to obtain a genomic test is allocated to the contributor in step 148. Whether the contributor actually obtains the free genetic test may depend on a random factor, for example by allocating a predetermined number of test randomly to the contributor or on other conditions.

The aspect of free or subsidized tests can be realized as follows: the health data provider provides funds for free or subsidized tests, for example by electronic payment to a test provider. The tests are then channeled to the test provider. In one example, the test provider has a list on its computer system, which lists the kind of test and the person with whom the test is associated with. The entries of the list are updated when the health data provider makes a payment for the test or pays a subsidy for the test.

A person registered with the web platform of the health data provider can then request an appointment for the free or subsidized test from the test provider by way of inputting contact data in a user interface.

A contributor of information is able to fine tune the extent that the contributor is willing to share according to three digital gene assets, which the contributor provides to the web platform over a user interface. These three digital gene assets are listed below. On the one hand, the digital gene assets take into account the willingness or ability of the contributor to provide data and, on the other hand, they also affect the reward being provided to the contributor. The digital gene assets also facilitate the search in the provided data.

The user interface of the web platform allows the contributors to set the digital gene assets at the time of registration and to modify the digital gene assets after registration. In one example, the contributor is rewarded with a pre-defined amount of gene credits for selecting a digital gene asset. By way of example, there can be three digital gene assets, which are listed below.

### 1. “Sign-up”

Sign up as a contributor and earn gene credits

### 2. “Update”

Update health information on the contributor's account

### 3. “Manage”

Tick on willingness to exchange information.

The willingness to exchange information can be expressed by the below mentioned factors, which the contributor can choose to elect or not to elect:
genetic data (provide raw data report),
molecular diagnostics of infectious diseases
agree to participate in a research project
agree to participate in a clinical trial
provide an existing specimen, such as blood, biopsy or other
provide updated or new specimen (resampling)
agree to answer questions from recipient, such as questions from a predetermined set of questions
link with members to share existing information
share radiology reports or radiology procedures, such as X-ray, CT-scan, MRI, mammography
share general laboratory reports
electronic medical record (EMR)
EMR on the health data provider's web site
other investigations
link with members with or without assets. This can be provided as a premium category asset and can enable a recipient to establish a direct contact to the contributor
provide data from portable or wearable devices for evaluation purposes

The genetic data can refer, among others, to biochemical genetics, molecular genetics, clinical cytogenetics, preimplantation genetic screening and non-invasive prenatal testing.

An enrolment to the clinical trial is available for contributors through a web site “Clinical Trial, Enroll” and for researchers through a web site “Research, Enroll”. Subsequent arrangements can be defined by the relevant parties.

In the case of sharing an existing specimen or providing a new specimen, the web site provides a means for arranging a shipping between the different parties.

In a further step, a verification of the user registration is carried out. By way of example, this can be done by using an SMS message, such as SMS TAC or e-mail verification. Herein, a TAC refers to a “transaction authorization code”. A user enters their telephone number in a personal profile and is then able to request a TAC, which is then sent to the user via SMS.

FIG. 14 illustrates a registration of a recipient at the health data provider 29 and a mechanism to provide benefits to the recipient.

In a first step 150, the recipient enters registration data. In a further step 151, the recipient pays a recipient registration fee, which varies according to an intended subscription category. A subscription category is determined in a next step 152. According to one alternative, the subscription category is chosen depending on the paid fee. According to another alternative, a recipient first chooses the subscription category and/or the sum corresponding to the subscription category and registration proceeds only when the amount required for the subscription category has been paid.

In a further step 153, a search period, such as for example 1 month, 3 months or 1 year, is allocated according to the subscription category. In particular, the search can refer to an “unlimited search” in which all of a predefined set of search categories can be searched. For example, the recipient can have access to a database of the health data provider for a fixed time period, such as two months, one year etc.

In a further step 154, a predetermined number #L of request links is allocated to the recipient, depending on the subscription category.

Furthermore, in a further step 155 a permission to ask a predetermined number of #Q anonymous questions is allocated to the recipient. Thereby, the recipient can ask questions to contributors anonymously, for example for the purpose of a survey or a questionnaire.

The query facilities are explained in more detail below. A recipient can do anonymous data mining through filter and search options according to a subscription model or “package”. The number of links that can be sent is pre-defined at the time of payment.

The recipient or consumer of the information is provided with a database query permission to query the database provided through the enrolled participants or subjects. The extent of the query permission can vary according to a chosen subscription model of the recipient. This can include selecting data of participants for which a requested information is available, such as whole exome tests, radiology scans etc.

The anonymous data mining can include, among others a search and filter for contributor data, such as genetic data, health data and miscellaneous data, such as demographic, lifestyle and nutrition data. The contributor data is provided by the contributor over the web interface, for example as part of the abovementioned registration step 141. Furthermore, the contributor data can also be provided by other parties to which contributor data is available, such as the test provider or the mass enrolment center. In general, the retrieval of contributor data from other parties requires an authorization by the contributor.

The data of the contributors which can be searched by the recipients can include, among others:
demographic profile, such as age, gender, location, profession
health status: diseased or healthy
diagnosed disease
medical history
history of allergy
medications
genetic report available
digital gene asset willingness

The anonymous data mining ca furthermore comprise an anonymous questionnaire to search and filter contributors.

As a further feature, the anonymous data mining can comprise a facility for linking and exchanging data for the transaction of data research and trials

Depending on the subscription category or package the recipients have furthermore access to an anonymous questionnaire to search and filter contributors. Among others, this questionnaire provides a possibility to query for genetic data, such as raw genetic data and genetic data reports.

According to another feature, the recipient can ask questions to contributors anonymously, for example for a survey or a questionnaire. With this feature, the application expands to data mining for the industry.

The recipient is provided with a second query permission which allows to query the subjects. For example, the querying of the subjects can be carried out with a predefined query mask or filter using a communication mechanism provided by the web platform of the health data provider, such as a message box and notification mechanism.

Furthermore, also consultants and third-party verifiers can register with the health data provider.

The web platform provides a randomized selection of third-party verifiers. The randomized selection can provide a more objective and unbiased verification as compared to a selection of third-party verification by the contributors themselves.

At the time of registration of a consultant at the web platform, the consultant agrees to three categories: (1) Verify data, (2) On-line consultation and (3) Booking of an appointment. For the purpose of booking appointments, the profile of the consultant is made available to others, in particular to contributors.

Through the registered consultants, contributors and recipient obtain access to a global database of clinicians they can link with for consultations. The global database can provide a seamless connection across geographies. At the same time, it provides a health care service on the web portal.

The web platform can provide a direct partnership model with retailers, which is illustrated below in the flow diagrams of FIGS. 15 to 20.

The partnership model can provide the following benefits, among others:
access to a large number of customers of the health data providers web platform
disbursement of discount vouchers and an easy access platform.
potential to enroll in a discount back program.
increase sales with an easy on-line partnership
recurring sales environments for the merchant where customers return to use utilize the discounts.
back end integration for monitoring of sales and/or partnership through contracts.

According to a first model, a direct partnership with retailers for Products is provided or with or without discount. According to this first model, the vendors promote their products through the health data provider's account. As a partnership contribution, the vendors provide a percentage discount to the health data provider, a percentage of which is passed back to the customers while retaining the fees. The customers use equivalent value in gene credits to avail their discounts and purchase the vendor's products.

The web platform of the health provider implements a “discount back program” whereby registered users get discounts and/or gene credits with each transaction with a vendor. Furthermore, retailer promotions are offered with discounts and gene credits for each purchase.

In particular, the promotions can include retailer promotions and redemptions of gene credits for products and services such as genomic tests, wellness tests, tumor tests, whole genome and exome retrieval/sequencing, health screening tests. Gene credits can be purchased as well and the gene credits can be exchanged for discount vouchers from a list.

The web platform provides a raw genetic data report for the contributors. Furthermore, the web platform provides a feature for linking with members and share an existing profile.

The registered contributors can receive a health consultation from a database of consultants, which can be counsellors, clinicians or pathologist, among others. The web platform also provides on-line consultations by a global data base of professionals.

In a further aspect, the web platform provides an automated system to manage and keep track of contributor spending and the gene credits balance of the contributors. The automated system provides a filter and a search based on specified criteria, as well as a “select all contributors” function to distribute the gene credits to the filtered accounts.

Furthermore, the automated system can generate a statement of account for monthly reporting for all contributors or based on filter and search options.

FIG. 15 illustrates a vendor commission program method using vouchers.

In a first step 158, the health data provider acquires bulk discount vouchers from one or more vendors. In a next step 159, the health data provider allocates vouchers to registered contributors according to their subscription category and/or other criteria. Moreover, in a further step 160, the health data provider allocates gene credits, which can be used to buy discount vouchers. In a further step 161, the contributors use the discounts to buy items from the vendor. In a further step 162, the health data provider receives a commission program from the vendor. In particular, the commission program can amount to a predetermined percentage of a price of the purchased items.

FIG. 16 illustrates a product purchase from a vendor associated to the health data provider.

In a first step 164, a contributor updates a contributor health information on their personal account. In a further step 165, the contributor accesses a vendor website and, in a next step 166, uses discount vouchers received from the health data provider to purchase items from the vendor.

In a further step 167, the contributor receives a purchase discount from the vendor. Furthermore, the contributor receives gene credits in a further step 168. In a next step 169, the contributor uses gene credits to purchase a voucher.

FIG. 17 shows an allocation of discount vouchers from the health data provider to the contributors.

In a first step 172, the health data provider purchases discount voucher from a vendor. In a further step 173, the health data provider allocates discount vouchers to registered contributors. Furthermore, the health data provider also provides links to vendors in step 175.

By way of example, the web platform can provide links to the vendors as part of a “gene market”, which is a platform or web portal where contributors use their discount voucher and gene credits to make purchases. Furthermore, merchants or vendors who wish to advertise their products and services are listed in the gene market. This listing is subject to a selection. For example, the listing of vendors can be limited to those vendors who have submitted an agreement to the health data providers. Or it can be limited to vendors who have paid a fee to be displayed on the gene market and subject to approval by the health data provider.

The gene market provides access to the following good and services, among others:
general merchandise. A link to a merchant or vendor platform is provided
nutraceutical. A link to a health supplement merchant is provided.
health and tourism. A link to a travel fair and health expo is provided.
health insurance. A link to an insurance provider is provided.
health tests: A list of tests is provided.
personalized genomics
solid tumor, clinical exome or whole exome examination
health screening packages.

In a step 176, the contributors purchase items from the vendors. In a further step 177, the vendor sends a purchase report to the health data provider. By way of example, this process can be triggered automatically after a predetermined amount of sold items and/or after a predetermined time period.

In a further step 178, the health data provider automatically computes a cashback value based on the received purchase reports and, in a next step 179, allocates vouchers and gene credits to the contributors based on the previously computed cashback value.

FIG. 18 shows an establishment of an agreement between a health data provider and a vendor and a cash flow between the vendor and the health data provider resulting from the agreement.

In a first step 182, the health data provider submits an agreement to the vendor. For example, the agreement may include a condition according to which the health data provider provides a link to the vendor and allocates vouchers specific for that vendor and a proportion of the vouchers and also a cashback that the vendor provides in return for items and/or services purchased via the web platform of the health provider and/or using the vouchers of the health provider. In particular, the conditions of the agreement or contract can be evaluated automatically and/or a compliance with the agreement conditions can be evaluated automatically.

In a further step 183, the vendor provides a fixed percentage of the sales to the health data provider. Herein, “sales” refers to a monetary value that the vendor receives for purchased items or services by way of electronic transmission, wherein the items are purchased directly via the web platform of the health data provider or by means of tokes such as vouchers received from the health data provider.

In a step 184 the health data provider allocates vouchers for purchasing items from the vendor to registered users which are registered over the web platform of the health data provider. In particular, these can be allocated to users of the category “contributor” or “recipient”.

In a further step 185, the health data provider provides vendor specific links to the vendor and, optionally, advertisements for the vendor. In a simple example, the advertisements can be provided by a clickable image which is associated with the web link. Furthermore, the advertisement can also comprise videos, interactive product catalogues or other interactive content.

In a step 186, the vendor sends a fixed percentage x % of the sales as sales participation to the health data provider. The sending occurs by way of electronic transmission and may involve an internet service for financial transactions such as a payment provider.

In a further step 187, the health data provider allocates gene credits based on the previous sales or purchases of the vendor. In a step 188, the health data provider also allocates discount vouchers based on the sales.

FIG. 19 shows a further cash flow between the vendor and the health data provider resulting from the agreement.

In a step 190, the vendor submits an agreement to the health data provider. In particular, the agreement can be submitted electronically using the web platform of the health data provider.

In a step 191, the vendor provides a fixed discount of x % of the sales to the health data provider, for example by way of electronic transmission of monetary units. Furthermore, in a step 192 the vendor provides y % initial discount to the health data provider. In one example, the initial discount refers to the agreement and is provided when the business agreement between the health data provider and the vendor is established. In a further example, an initial discount is provided on a per customer basis for every new customer.

In a step 193, the health data provider passes on the discount to the contributor in form of vouchers and/or gene credits.

In a further step 194, contributors purchase items from the vendor. In a step 195, the health data provider receives fees from the vendor, based on the agreement and, in a step 196, the health data provider passes on benefit to the contributor from the free received from the vendor in the form of vouchers and/or gene credits.

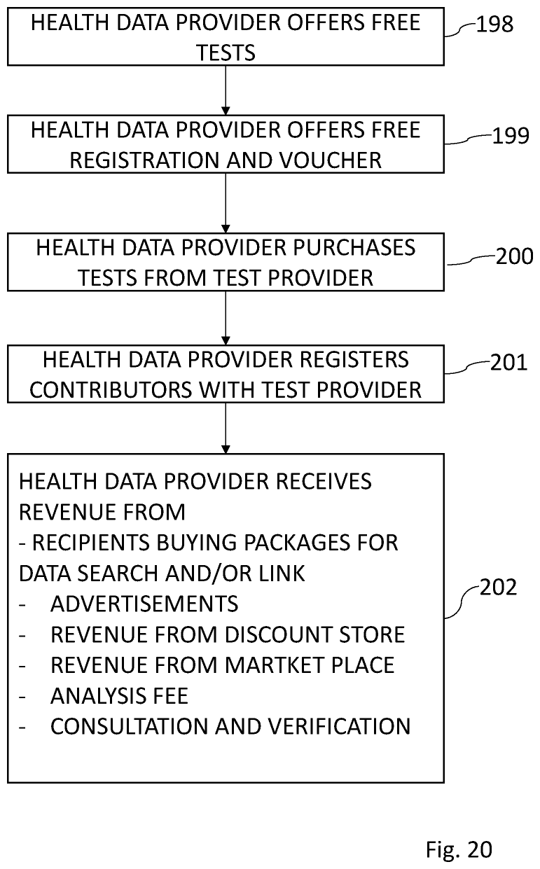

FIG. 20 illustrates an interaction between the health data provider and the vendor in a vendor cashback procedure.

In a step 198, the health data provider offers free tests for registered customers. This can be done for example by notifying the registered customers of the free test and sending a token to the test provider, which contains data indicative of the test paid for and the customer entitled to the test. By using the token, the test provider can verify the entitlement of the respective customer to the free test. The token can be transmitted or read optically or via a wireless connection or communication channel such as a near field connection or a WIFI connection. In a simple embodiment, the token is a text message. Furthermore, the customer is notified of the free test, for example by a status update of the customer profile or by sending a message.

In a step 199, the health data provider offers a free registration and vouchers.

Furthermore, in a step 200, the health data provider purchases free tests from a test provider. In general, the order in which the steps are performed can vary from embodiment to embodiment. For example, the step 200 of purchasing the tests can also be performed before the step 198 of offering the tests to registered customers.

A further usage of gene credits as reward comprises assigning or allocating gene credits to contributors who can refer a friend. For example, a fixed percentage of the registration fee of the referred person, such as 10%, can be awarded to the contributor.

According to one example, the contributor is awarded 10% of the registration fees as Gene credits for every new customer or “friend” referred to who then registers and pays at least a minimum sum such as USD 5 for the registration.

In general, gene credits are assigned to asset providers, such as recipients and contributors. According to one embodiment, the gene credits, or a fixed portion of them, is algorithmically associated with shares of value generated from the entirety of the assets.

Within the framework provided by the health data provider, the gene credits can be used as a kind of currency and provide the following benefits, among others:

1. Gene Credits can provide discounts, by using gene credits to avail discounts on e-shopping platforms.
2. Gene Credits can provide redemptions on products and services provided by the service provider.
3. Gene Credits can be used as cash equivalent to pay for genomic tests, such as the following types of tests:
(i) personalized genomics
(ii) clinical exome
(iii) whole exome
(iv) solid tumor

Furthermore, the gene credits in an account can be used for discounts at e-commerce merchants as a discount value on the merchant's e-shopping platform. Users which are registered at the web platform of the health data provider can redeem for product and services at a marketplace web interface. Among others, gene credits can be used to buy discount vouchers from listed sites and to get direct redemptions for various products and services.

According to a further embodiment, the mass enrolment center is the entity which receives the payments of asset users and the health data provider receives the payments from the MEC. Thereby, the health data provider becomes a second-grade trustee and the MEC a first-grade trustee.

Product items for which the health data provider provides discounts and promotions are, among others:

General merchandise, nutraceuticals, health and tourism products and services, general health care items, health insurance and health tests and genomic tests.

A discount store concept, which is of value for partner merchants and which can provide contributors with a continuous incentive to shop at partner sites or vendors, comprises the following steps:

The health data provider has a predefined monthly budget to build a discount buyback program. The monthly budget is allocated to buy merchant vouchers. The health data provider buys bulk vouchers from merchants to avail best rates for purchase of vouchers from vendors.

Every contributor has a unique and secure personal account at the health data providers web platform. The health data provider's platform provides every contributor with gene credits equivalent to cash upon registration.

In a given example, a contributor pays a small registration fee, such as USD 5, and receives USD 2 merchant discount voucher and 100 Gene Credits equivalent USD 10 in their account. In one example, the gene credits are tied to a currency or to a mix of currencies. For example, a conversion rate can be fixed as 10 gene credits=1 USD.

For the first few months upon launch the health data provider offers free Registration and an additional of USD 1 free voucher for proposed various merchant partners. Furthermore, the health data provider enrolls in a cash back program with vendors. This allows to constantly buy discount vouchers from the vendor or the vendors.

As a further feature, a mobile app is provided. This app allows contributors and recipients registered on the site to search link and filter with people. Furthermore, the app provides networking opportunities such as the creation of chat groups with people with similar interests or requests. For example, the contributors could link to a global Down syndrome society.

As a further feature, the web platform provides a personalized electronic record. Often, electronic records are controlled by hospitals of clinicians. By contrast, the web platform can provide its own personalized medical record for contributors with reminders for tests or screens, health advice etc. Even if a person travels to various places his record is uniquely maintained by him or her. It is consolidated information.

According to a further embodiment, a subscriber can appoint an administrator or the health data provider to search for a relevant subject/contributor. The admin of the health data provider searches and filters and contacts contributors and the mass enrolment center (MEC) for consent and negotiation of rate. After finalizing the list of contributors, the admin provides the recipient with the list for online payment. The MEC is also referred to as “healthcare partner.” According to a further embodiment or feature, the healthcare partner (MEC) can post information about deidentified anonymized consented data available with the healthcare partner.

The recipients can filter and search and view available such databases. If the recipient wishes to acquire this data, they send a link request to the admin who then connects with the healthcare partner to arrive at a negotiated deal.

According to a further embodiment or feature, which relates to an emergency response, a digital healthcare product for an emergency response is provided. A personal account of the health data provider has the healthcare record of each contributor. Furthermore, the digital healthcare product allows the contributor to empanel and authorize to relatives or friends who can access the contributor's healthcare information in case of an emergency.

The emergency friend registers as a new role on the site and has his/her own account. This account allows access to only the medical record of the Contributor in case of an emergency.

According to a further embodiment or feature, which relates to a personalized electronic medical record (EMR), the health data provider records a predefined amount of medical information of the Contributor. This functionality can be expanded by either adding on more features for medical history including uploading of reports. Alternatively, the health data provider can interact with a third party EMR provider who can customize a personalized EMR for each contributor. In this case the complete EMR of Contributor is stored on a third-party vault. The personalized EMR can be made active by customizing for reminders, testing, screening and other healthcare activities.

The subject matter of the present specification also comprises the following feature combinations, which can also be combined with other features of the present specification. For the sake of simplicity only, the following feature combinations are organized as an itemized list with dependencies.

When the items refer to communication parties, such as subscriber, third-party subject etc. this can also refer to the device of the communication party.

Item 1. A computer-implemented method for establishing a communication link for the automated exchange of health-related information between one or more subjects and a subscriber or between a mass enrolment center and a subscriber via a web platform, the method comprising
receiving data relating to a health data type and/or a genetic data type of subjects,
selecting a second communication partner, such as an information source a subject or an MEC by a first communication partner, subscriber on the basis of the received health data type and/or a genetic data type,
receiving a payment from at least one of the first communication partner and the second communication partner,
creating a temporary link site with a temporary site identifier, such as an IP address that can be assigned from a pool of available IP addresses,
forwarding the link site identifier to the first communication partner and to the second communication partner,
storing a first identifier, IP address, of the first communication partner and a second identifier, IP address, of the second communication partner,
providing a communication interface which allows the interchange of pre-determined messages, related to health data and/or genetic data, between the first communication partner and the second communication partner,
checking a termination condition and, when the termination condition is satisfied, terminating the link interface. Terminating means at least: no longer providing or disabling the communication interface under the link site identifier.
   Item 2. Method according to item 1, comprising:
releasing a personal identity of the second communication partner only after a payment has been received from at least the first communication partner or the second communication partner.

Automatic triggering of payment and commodity exchange after selecting predefined messages, hitting accept button, no individual free-text messages allowed.

Item 3. A computer-implemented method for the automated exchange of health-related information between one or more subjects and a subscriber via an information provider, the method comprising, at a site of the subscriber,
identifying, selecting, predefined query items, for retrieving health related data from the one or more subjects, e.g. predefined text messages,
sending an information request message to an information provider, e.g. mass enrolment center, the request message comprising a consent query and the predefined query items,
   at a site of the information provider, e.g. mass enrolment center,
receiving the request message,
determining whether consent is granted to send, anonymized, individual health data,
   if it is decided that consent is granted:
sending a—preferentially encrypted—data message to the subscriber, the data message comprising the individual health data, wherein a payment request to the subscriber is automatically triggered by a predefined event and the sending of the data message to the subscriber is conditional on a payment provided by the subscriber in response to the payment request.
   Item 4. The method according to item 3, wherein the step of deciding whether consent is granted comprises exchanging consent information with a user device of subject.
   Item 5. The method according to items 3 or 4, wherein the step of deciding whether consent is granted comprises querying a database of the health data provider for consent related data.
   Item 6. The method according to one of the items 3 to 5, wherein the predefined event that triggers the payment request comprises the selection of predefined messages, and the activation of an accept button.

In the following, a subscriber site can also be provided by a simple message receiving program on a user device to which message can be sent using a destination address and does not need to be a web site.

Item 7. The method according to one of items 3 to 6, further comprising
at a payment site
sending a payment request to the subscriber site,
receiving payment data from the subscriber site,
confirming the payment,
   if the payment is confirmed
send a payment acknowledgment message to the information provider,
   at the information provider
receiving the payment acknowledgment message and, upon receipt of the payment acknowledgment message, sending, transmitting, the data message to the subscriber.

Successive identification of information relevant to the subscriber. A successive refining can be provided by a program providing a set of predefined questions or choices that allow to narrow down the scope of the information.

Item 8. The method according to one of the preceding items 3 to 7, further comprising
retrieving predefined criteria for successively refining a type of information to be queried from a subject,
displaying the predefined criteria to the subscriber through elements of a graphical user interface, wherein the predefined event comprises a matching of the predefined criteria with a requirement provided by the subscriber.
   (alternative: the query message can also be sent to the information provider directly and payment provider will only be notified once the consent is obtained. Furthermore: payment can be made in advance, in which case there would be a request message whether payment has been made/subscription is currently active.
   Item 9. A system for the automated exchange of health-related information between one or more subjects and a subscriber via an information provider, the system comprising:
a subscriber user device which is operative
   to identify, selecting, predefined query items, for retrieving health related data from the one or more subjects, e.g. predefined text messages,
   to send an information request message to an information provider, e.g. a mass enrolment center or a web platform, the request message comprising a consent query and the predefined query items,
      a data provider server device which is operative
to receive the request message,
to determining whether consent is granted to send, anonymized, individual health data, and, if it is decided that consent is granted:
to send an, encrypted, data message to the subscriber, the data message comprising the individual health data, wherein a payment request to the subscriber is automatically triggered by a predefined event and the sending of the data message to the subscriber is conditional on a payment provided by the subscriber in response to the payment request.
   Item 10. A computer-readable storage medium, which can include multiple physical carriers or cloud storage, comprising instructions which, when executed by a system for the automated exchange of health-related information between one or more subjects and a subscriber via an information provider, cause respective computers of the computer system at a site of the subscriber
to identify, selecting, predefined query items, for retrieving health related data from the one or more subjects, e.g. predefined text messages,
to send an information request message to an information provider, e.g. mass enrolment center, the request message comprising a consent query and the predefined query items, at a site of the information provider, e.g. mass enrolment center
to receive the request message,
to determine whether consent is granted to send, anonymized, individual health data, and, if it is decided that consent is granted:
to send an, encrypted, data message to the subscriber, the data message comprising the individual health data, wherein a payment request to the subscriber is automatically triggered by a predefined event and the sending of the data message to the subscriber is conditional on a payment provided by the subscriber in response to the payment request.

The transaction involving: consent proxy for mass enrolment center, which enables communication with several selected information exchange channels at the same time. There is a one-to-one relationship between subjects and subscribers. Subjects or individuals could be identified at subscriber site or at information provider site.

Item 11. A computer-implemented method for the exchange of health-related information between one or more subjects and a subscriber via an information provider, the method comprising at a subscriber site
identifying health information criteria,
identifying a set of subject identifiers based on the health information criteria,
sending an information request message for obtaining medical information from the one or more subjects corresponding to the subject identifiers, e.g. bulk information exchange, at a proxy server site
receiving the information request message,
obtaining consent information for individual health data of the one or more subjects identified by the information request message,
   e.g. by matching the provided information in a database against stored consent criteria of the individuals, which is faster than obtaining the consent from each individual, which may not be online,
   after a pre-defined information release event
retrieving health information from the one or more subjects,
sending a health information message to the subscriber, the health information message comprising health data that matches to the consent data, e.g. the health information message may comprise only information for a subset of the individuals or only limited information and the message may indicate this explicitly.
   Item 12. The method of item 11, comprising
verifying payment data for the information request message,
if the payment verification is positive triggering the predefined information release event.
   Item 13. The method of item 11 or item 12, wherein the retrieval of health information from the one or more subjects comprises
initiating separate information exchange services with the one or more subjects, at the health information providing for the exchange of health information, the information exchange services comprising
providing pre-defined selectable data,
presenting the pre-defined selectable data to the one or more subjects.

Payment can be obtained at the moment of transaction or payment can be provided in advance. Data can be provided as anonymized individual data sets or in the form of aggregated data. If the information is too different between individuals and therefore cannot be aggregated, it is provided as anonymized individual data sets. The subscriber must agree to refrain from attempting to trace back the anonymized information. Resources can be provided a dedicated hardware, or it can be provided by cloud computing, which can involve sharing across the web or across different servers or across other resources. The identified group of individuals can be stored for later use to enable follow up information, e.g. how long does the hip prosthesis last.

Item 14. A system for the automated exchange of health-related information between one or more subjects and a subscriber via an information provider, the system comprising:
a subscriber user device which is operative
   to identify health information criteria,
identifying a set of subject identifiers based on the health information criteria,
to send an information request message for obtaining medical information from the one or more subjects corresponding to the subject identifiers, e.g. bulk information exchange,
a proxy server device which is operative
   to receive the information request message,
   to obtain consent information for individual health data of the one or more subjects identified by the information request message,
      e.g. by matching the provided information in a database against stored consent criteria of the individuals, which is faster than obtaining the consent from each individual, which may not be online,
      after a pre-defined information release event
to retrieve health information from the one or more subjects,
to send a health information message to the subscriber, the health information message comprising health data that matches to the consent data, e.g. the health information message may comprise only information for a subset of the individuals or only limited information and the message may indicate this explicitly.
   Item 15. A computer-readable storage medium, which can include multiple physical carriers/cloud storage, comprising instructions which, when executed by a system for the automated exchange of health-related information between one or more subjects and a subscriber via an information provider, cause respective computers of the computer system
   at a subscriber site
to identify health information criteria,
to identify a set of subject identifiers based on the health information criteria,
to send an information request message for obtaining medical information from the one or more subjects corresponding to the subject identifiers, e.g. bulk information exchange, at a proxy server site
   to receive the information request message,
   to obtain consent information for individual health data of the one or more subjects identified by the information request message,
      e.g. by matching the provided information in a database against stored consent criteria of the individuals, which is faster than obtaining the consent from each individual, which may not be online,
after a pre-defined information release event
   to retrieve health information from the one or more subjects,
   to send a health information message to the subscriber, the health information message comprising health data that matches
to the consent data, e.g. the health information message may comprise only information for a subset of the individuals or only limited information and the message may indicate this explicitly.

A robot for doing “the information exchange” for the subscriber.

Item 16. A method for the exchange of health-related information between one or more subjects and a subscriber via an information provider, the method comprising
initiating an information exchange with a subject, querying repeatedly until a subject is available/requesting a time when the subject is available,
exchanging consent data with the subject, repeating the steps of
sending a pre-defined information request to the subject,
waiting for a pre-defined response message, which includes no free-text, to the information request,
   if the pre-defined response message is received
storing the content of the pre-defined response message for later use.
   Item 17. A system for the automated exchange of health-related information between one or more subjects and a subscriber via an information provider, the system comprising:
a proxy server device, the proxy server device being operative
to initiate an information exchange with a subject, querying repeatedly until a subject is available or requesting a time when the subject is available,
   to exchange consent data with the subject, to repeat the steps of
sending a pre-defined information request to the subject,
waiting for a pre-defined response message, no free-text, to the information request,
   and, if the pre-defined response message is received
to store the content of the pre-defined response message for later use.
   Item 18. A computer-readable storage medium, which can include multiple physical carriers/cloud storage, comprising instructions which, when executed by a system for the automated exchange of health-related information between one or more subjects and a subscriber via an information provider, cause respective computers of the computer system
to initiate an information exchange with a subject, querying repeatedly until a subject is available/requesting a time when the subject is available,
to exchange consent data with the subject, to repeat the steps of
sending a pre-defined information request to the subject,
waiting for a pre-defined response message, no free-text, to the information request,
   and, if the pre-defined response message is received
to store the content of the pre-defined response message for later use.
   Item 19—An arrangement for encrypted exchange of personal medical and financial data, the arrangement comprising
a subscriber computing device, operable to receive the health-related data and the identity information data via a communication interface,
a web platform server device, operable to store health related data and identity information data of a multitude of subjects and operable to provide an encrypted communication channel between the subscriber computing device and web platform server device,
a payment provider server device, operable to receive a payment information from the web platform server device and/or from the subscriber computing device, and to provide a payment signal 3 to the web platform server device 28, wherein the providing of the encrypted communication channel by the web platform server device comprises:
   creating a temporary link site with a temporary link site identifier and a computer memory link location, forwarding the link site identifier to the web platform server device and to the subscriber computing device,
the temporary link site providing a communication interface which allows the encrypted interchange of pre-determined messages between the web platform server device and the subscriber computing device, using the computer memory link location for releasing the health-related data to the subscriber computing device,
   wherein the web platform server device, upon reception of the payment signal from the payment provider server device, automatically initiates a transmission of the identity information data of at least one subject from the web platform server device to the subscriber computing device.

### REFERENCE

3 payment signal
5 MEC computer
7 data transfer message
8 request message
9 request message
10 Medical information exchange system
11 first user device
12 subject
13 first server device
14 mass enrolment center/health data provider/mass enrolment center computing device
15 second user device
16 subscriber/subscriber computing device
17 second server device
18 payment platform/payment provider server device
19 health provider database
19/1 health related data
19/2 identity information
20 payment platform database
21 information request message
22 response message
23 query server
24 query provider
25 database
26 third-party device
27 third party
28 web platform
29 health data provider
30-43 method steps of FIG. 4
51-61 method steps of FIG. 5
72-86 method steps of FIG. 6
87-102 method steps of FIG. 7
103-115 method steps of FIG. 8
100, 100′, 100″ health information exchange system
119 gene credits
120 gene tests
121 GUI input mask
122 GUI input mask
123 communication infrastructure
124 admin
125 link site
126 IP address link site
127 IP address first communication partner
128 IP address second communication partner
129 computer memory link location
130 associated service provider
131 communication channel
132 communication channel
133 communication channel
134 communication channel
135 communication channel
136 communication channel
137 communication channel
138 communication channel
139 communication channel
140-148 method steps FIG. 13
150-155 method steps of FIG. 14
158-162 method steps of FIG. 15
164-169 method steps of FIG. 16
172-179 method steps of FIG. 17
182-188 method steps of FIG. 18
190-196 method steps of FIG. 19
198-202 method steps of FIG. 20
203 communication channel
204 communication channel

1. A system for encrypted exchange of personal medical and financial data, the system comprising:
a mass enrollment center computing device, operable to store health-related data and identity information data of a multitude of subjects,
a subscriber computing device, operable to receive the health-related data and the identity information data via a communication interface,
a web platform server device, operable to provide an encrypted communication channel between the subscriber computing device and the mass enrollment center computing device, the providing of the encrypted communication channel by the web platform server device comprising:
creating a temporary link site with a temporary link site identifier and a computer memory link location,
forwarding the link site identifier to the mass enrollment center computing device and to the subscriber computing device,
providing, by the temporary link site, a communication interface that allows the encrypted interchange of pre-determined messages between the mass enrollment center computing device and the subscriber computing device, and
using the computer memory link location for releasing the health-related data to the subscriber computing device, and
a payment provider server device, operable to receive a payment information from one of the mass enrollment center computing device and/or from the subscriber computing device, and to provide a payment signal to the web platform server device,
wherein the web platform server device is operable to automatically initiate a transmission of the identity information data of at least one subject from the mass enrollment center computing device to the subscriber computing device upon receipt of the payment signal from the payment provider server device.

2. The system according to claim 1, wherein the communication interface is operable to provide an automatic verification of matching of the health-related data with a predetermined message of the subscriber computing device, upon which the subscriber computing device is triggered to send a payment information to the payment provider server device.

3. The system according to claim 1, wherein the web platform server device is operable to store a first identifier of the mass enrollment center computing device and a second identifier of the subscriber computing device, and wherein the communication interface is maintained between the first identifier and the second identifier.

4. The system according to claim 1, wherein the web platform server device is operable to delete the temporary link site and the computer memory link location after transmission of the identity information data of the least one subject from the mass enrollment center computing device to the subscriber computing device.

5. The system according to claim 1, wherein the web platform server device is furthermore operable to
receive identification data of a referred person from a referring subject, and
award, upon registration of the referred person, gene credits to an account of the referring subject.

6. The system according to claim 1, wherein the web platform server is furthermore operable to
attribute a third-party verification service to one or more registered subjects, wherein the attribution of the third-party verification service is randomized, and
cause the third-party verification service to verify health-related data of the one or more registered subjects, wherein the health-related data is filtered according to exchange categories that are individually specified by the one or more subjects.

7. The system according to claim 1, wherein the web server computing device is furthermore operable to
receive a health exchange category from each of one or more subjects,
store the health exchange category of each of the one or more subjects,
provide access to health-related data according to the stored health exchange categories, the health exchange categories being selected from genetic data relating to biochemical genetics, molecular genetics, clinical cytogenetics, preimplantation genetic screening or non-invasive prenatal testing, molecular diagnostics of infectious diseases, clinical trials, sharing of an existing specimen, providing a new specimen, radiology procedures, laboratory reports, electronic medical records and health-related data retrieved from wearable devices.

8. The system according to claim 1, wherein the web server computing device is furthermore operable to
store health-related data of the at least one subject,
receive a first evaluation of the health-related data based on the health-related data and based on at least one exchange category chosen by the at least one subject, and
adjust, after receiving the first evaluation, the first evaluation based on a demand for the health-related data.

9. A method for encrypted exchange of personal medical and financial data, the method comprising:
storing health-related data and identity information data of a multitude of subjects at a mass enrollment center computing device, receiving the health-related data and identity information data at a subscriber computing device via a communication interface,
providing an encrypted communication channel between the subscriber computing device and the mass enrollment center computing device by a web platform server device, further comprising:
creating a temporary link site with a temporary link site identifier and a computer memory link location;
forwarding the temporary link site identifier to the mass enrollment center computing device and to the subscriber computing device;
providing, by the temporary link site, a communication interface that allows the encrypted interchange of pre-determined messages between the mass enrollment center computing device and the subscriber computing device;
using the computer memory link location for releasing the health-related data to the subscriber computing device;
receiving a-payment information at a payment provider server device, and providing a payment signal to the web platform server device, and
automatically initiating, upon receipt of the payment signal, a transmission of the identity information data of at least one subject from the mass enrollment center computing device to the subscriber computing device.

10. The method according to claim 9, further comprising providing an automatic verification of a matching of the health-related data with a predetermined message of the subscriber computing device.

11. The method according to claim 9, further comprising storing a first identifier of the mass enrollment center computing device and a second identifier of the subscriber computing device, wherein communication is maintained between the first identifier and the second identifier.

12. The method according to claim 9, further comprising deleting each of the temporary link site and the computer memory link location is deleted after the transmission of the identity information data of the least one subject from the mass enrollment center computing device to the subscriber computing device.

13. The method according to claim 9, further comprising:
receiving identification data of a referred person from a referring subject, and
awarding, upon registration of the referred person, gene credits memory equivalent to monetary value to an account of the referring subject.

14. The method according to claim 9, further comprising:
attributing a third-party verification service to one or more registered subjects, wherein the attribution of the third-party verification service is randomized, and
causing the third-party verification service to verify health-related data of the one or more registered subjects, wherein the health-related data is filtered according to either of exchange categories and digital gene assets, the either of the exchange categories and the digital gene assets being individually specified by the one or more subjects.

15. The method according to claim 9, furthermore comprising:
receiving a health exchange category from each of one or more subjects,
storing the health exchange category of each of the one or more subjects,
provide access to health-related data according to the stored health exchange categories, the health exchange categories being selected from genetic data relating to biochemical genetics, molecular genetics, clinical cytogenetics, preimplantation genetic screening or non-invasive prenatal testing, molecular diagnostics of infectious diseases, clinical trials, sharing of an existing specimen, providing a new specimen, radiology procedures, laboratory reports, electronic medical records and health-related data retrieved from wearable devices.

16. The method according to claim 9, further comprising:
storing health-related data of the at least one subject, receiving a first evaluation of the health-related data based on the health-related data and based on at least one exchange category chosen by the at least one subject, and
adjusting, after receiving the first evaluation, the first evaluation based on a demand for the health-related data.

17. A computer-readable digital storage area comprising instructions which, when executed by a computer system provides the steps of a method according to claim 9.
